(19) United States
(12) Patent Application Publication
(10) Pub. No.: US 2026/0272317 A1
(43) Pub. Date: Sep. 17, 2026

(54) A STACKED-DESIGN MULTI-MODE CONFORMABLE EPIDERMAL SENSOR

(71) Applicant: BOARD OF REGENTS, THE UNIVERSITY OF TEXAS SYSTEM, Austin, TX (US)

(72) Inventors: Nanshu LU, Austin, TX (US); Sarnab BHATTACHARYA, Austin, TX (US); Philip TAN, Austin, TX (US); Pulin WANG, Austin, TX (US); Francesca SANTUCCI, Austin, TX (US)

(21) Appl. No.: 19/168,130

(22) PCT Filed: Mar. 21, 2024
(86) PCT No.: PCT/US2024/020891

§ 371 Date: Sep. 23, 2025

Related U.S. Application Data
(60) Provisional application No. 63/454,195, filed on Mar. 23, 2023.

(51) Int. Cl. A61B 5/0255; A61B 5/00; A61B 5/01; A61B 5/02; A61B 5/021; A61B 5/257; A61B 5/282

(52) U.S. Cl. CPC A61B 5/0255; A61B 5/01; A61B 5/02028; A61B 5/02125; A61B 5/02141; A61B 5/257; A61B 5/282; A61B 5/721; A61B 5/725; A61B 2560/0214; A61B 2562/0219; A61B 2562/0271; A61B 2562/164; A61B 2562/166; A61B 2562/227

(57) ABSTRACT

A stacked-design multi-mode conformable epidermal sensor that, when worn on an epidermis of a human proximate the heart (e.g., chest), is capable of synchronously/continuously monitoring electrical activity and mechano-acoustic activity of a cardiovascular system.

### CROSS REFERENCE TO RELATED APPLICATION

This application claims priority to and benefit of U.S. provisional patent application Ser. No. 63/454,195 filed Mar. 23, 2023, which is fully incorporated by reference and made a part hereof.

### GOVERNMENT SUPPORT

This invention was made with government support under Grant no. ECCS2133106 awarded by the National Science Foundation and Grant no. N00014-20-1-2112 awarded by the Office of Naval Research. The government has certain rights in the invention.

### BACKGROUND

Cardiovascular diseases (CVDs) are the leading cause of death in the United States and burden the nation hundreds of billions of dollars each year. To reduce the fatality and social costs caused by CVD, wearable continuous cardiovascular monitoring devices and/or ambulatory blood pressure measurement (ABPM) devices may be required for timely diagnosis and treatment of CVD.

Specifically, hypertension is the most common and strongest risk factors for almost all different cardiovascular diseases. Over 30% of U.S. adults or a population of 75 million are hypertensive and only half of them have their blood pressure under control. Numerous studies have reported in the literature that using ABPM can significantly reduce the risks and cost of heart disease management. However, cardiologists are reluctant to prescribe ABPM to their patients due to the difficulties using current ABPM devices. In addition to BP, other crucial parameters for clinical monitoring include SV (Stroke Volume) and CO (Cardiac Output). SV quantifies the blood volume expelled by the heart per cardiac cycle, while CO measures the blood volume pumped by the heart within one minute. These metrics offer informative assessments of cardiac health, reflecting the heart's contractile capacity and compliance in accommodating the body's needs. This significance amplifies during physical exertion, where the body's increased oxygen demand underscores the importance of monitoring the heart's adaptive response to such demanding conditions.

Cardiovascular function may be monitored by sensing the electrical activity of the heart (e.g., electrocardiogram). In addition, cardiovascular function may be monitored by sensing mechanical or acoustic (i.e., mechano-acoustic) activity of the heart (e.g., phonocardiogram, seismocardiogram, and ballistocardiogram).

Sensing electrical and mechano-acoustic activity provides complementary information. For example, electrical activity may provide information regarding myocardial conduction, while mechanical activity may provide information regarding myocardial contraction.

Traditionally, various types of equipment have been used for measuring electrical activity and mechano-acoustic activity of a cardiovascular system. For example, an electrocardiogram (ECG) may be obtained using a wearable Holter monitor; a phonocardiogram (PCG) may be obtained using a stethoscope; a seismocardiogram (SCG) may be obtained using a digital accelerometer worn on the chest, and a ballistocardiogram (BCG) may be obtained using a swing bed or a force sensor placed on a weighing scale. Seismocardiography measures the vibrations of the chest generated by the heartbeat. While it offers valuable insights on cardiovascular health, its efficacy may be compromised by motion-induced artifacts.

Measuring a blood pressure (BP) of a cardiovascular system has traditionally required a sphygmomanometer, which uses a pressurized cuff. The inflation/deflation of the cuff makes beat-to-beat BP measurements impossible. Beat-to-beat BP measurements, however, are highly desirable for quickly assessing various condition associated with CVDs (e.g., heart disease, stroke, end-stage renal failure, and peripheral vascular disease).

To sense a beat-to-beat BP, an ECG sensor (worn on the chest) and a photoplethysmogram (PPG) sensor (worn on a finger) may be used in combination to measure the time that takes for a pulse pressure (PP) waveform to propagate through a length of the arterial tree. This approach is not practical for long term sensing because of the inconvenient sensor configuration. In addition, conventional silver/silver chloride (Ag/AgCl) gel electrodes may result in skin irritation and dehydration may degrade their performance if worn for extended periods. Furthermore, traditional systems for monitoring SV and/or CO in clinical settings, such as patient monitors based on impedencecardiogram (ICG), are bulky and stationary, making them unsuitable for ambulatory monitoring.

Recent research has shown that synchronous measurements of (i) the electrical activity of the heart (i.e., ECG) and (ii) local vibrations of the chest wall induced by the seismic motion of the heart (i.e., SCG) or whole-body movement caused by the ballistic forces on the heart (i.e., BCG) can be used to estimate a beat-to-beat BP and to predict SV/CO.

Conventional approaches for synchronous measurements of ECG and SCG (or BCG) are still challenged by reliability, accuracy, cost, accessibility, and/or comfort. For example, mounting a rigid accelerometer or rigid piezoelectric transducer on a human chest to measure SCG is uncomfortable and not practical for extended periods.

Conventional ambulatory devices for cardiac monitoring have disadvantages, including: 1) Only ECG is sensed which gives insight into the electrical activity of the heart but not the mechanical performance of the heart. In some cases, heart failure can still occur even if the ECG is not abnormal. Moreover, only the combined use of ECG and SCG enables the extraction of certain parameters, such as typical time intervals of the cardiac cycle, namely the Cardiac Time Intervals (CTI). 2) The available devices are rigid, bulky, not comfortable to wear, and socially stigmatizing which reduced patient compliance.

A need therefore exists for an integrated, wearable stacked-design multi-mode sensor that overcomes challenges in the art, some of which are described above.

### SUMMARY

Disclosed and described herein are embodiments of a device that employs multimodal sensing (e.g., ECG, SCG, PPG and temperature sensing), allowing the monitoring of both electrical and mechano-acoustic activity of the heart. PPG sensing is used to monitor the vasculature and estimate the BP; while the conjunct use of ECG and SCG allows the extraction of fundamental cardiovascular timing intervals. The disclosed embodiments of the device comprise a thin, light, skin conformal form factor that can be laminated onto the chest. It is soft and stretchable and is comfortable to wear. Furthermore, embodiments of the device combine cardiac and BP monitoring into a single ambulatory device having wireless streaming capability for real time cardiovascular monitoring.

Synchronized ECG, SCG and PPG sensing enables more holistic cardiovascular monitoring and offers the potential for beat-to-beat BP and cardiac performance tracking. Wireless streaming capability enables data offloading for real time data analysis, as well as long term storage and holds potential for real time diagnosis. Using multimodal sensing capabilities, different cardiac events such as QRS complex, valve openings and closing can be detected which enable the measurement of important cardiac time intervals like pre-ejection period (PEP) and left ventricular ejection time (LVET) which are indicators of cardiac health. Vascular timing intervals like pulse transit time (PTT) can also be extracted, which has high correlation with blood pressure (BP).

In one aspect, a stacked-design multi-mode conformable epidermal sensor is described. One embodiment of the sensor comprises a transparent first flexible, stretchable insulating substrate comprising a first side and a second side opposite the first side, wherein the first side adheres to an epidermis and the transparent first flexible, stretchable insulating substrate conforms to the epidermis; an electrocardiogram (ECG) sensor comprising one or more biocompatible electrodes, wherein each electrode forms an electrode pattern on the first side of the transparent first flexible, stretchable insulating substrate and each electrode is configured to flex and stretch with the transparent first flexible, stretchable insulating substrate to conform to the epidermis; a seismocardiogram (SCG) sensor, said SCG sensor, mounted on a second side of a second flexible insulating substrate, said second flexible insulating substrate having a first side that is substantially in contact with the second side of the transparent first flexible, stretchable insulating substrate; and electronics disposed at least partially on the second side of the second flexible insulating substrate. The electronics comprising at least a wireless communications interface, a central processing unit, an accelerometer used by the SCG sensor, the ECG sensor, and a power source, wherein components of the electronics are separated from one another on the second side of the second flexible insulating substrate and connected to one another using conductive (e.g., copper) traces configured to flex and stretch with the transparent first flexible, stretchable insulating substrate and/or the second flexible insulating substrate, and to conform to the epidermis, wherein the electronics are connected to the one or more biocompatible electrodes (e.g., graphite film electrodes) of the ECG sensor using a conductive material such as anisotropic conductive film (ACF) windows in the transparent first flexible, stretchable insulating substrate, or conductive material (e.g., ACF windows) in both the transparent first flexible, stretchable insulating substrate and the second flexible insulating substrate.

In some instances, the sensor may further comprise a photoplethysmogram (PPG) sensor disposed on the first side of the second flexible insulating substrate. The PPG sensor may comprise one or more light emitting diodes (LEDs) and one or more photodetectors (PD) that perform reflective photoplethysmography through the transparent first flexible, stretchable insulating substrate. The LED and PD electrically connected to the electronics disposed at least partially on the second side of the second flexible insulating substrate through the second flexible insulating substrate.

In some instances, the sensor may further comprise a temperature sensor. The temperature sensor may be disposed on the first side of the second flexible insulating substrate and connected to the electronics disposed at least partially on the second side of the second flexible insulating substrate through the second flexible insulating substrate.

In some instances of the stacked-design multi-mode conformable epidermal sensor, the transparent first flexible, stretchable insulating substrate may comprise a polyurethane film having an adhesive layer on the first side of the transparent first flexible, stretchable insulating substrate.

In some instances of the stacked-design multi-mode conformable epidermal sensor, the second flexible insulating substrate may comprise a polyimide flexible insulating substrate.

In some instances of the stacked-design multi-mode conformable epidermal sensor, the electronics may further comprise a second accelerometer. The second accelerometer may be used for one or more of motion compensation, cancellation of motion artifacts, orientation detection, and activity recognition. In some instances, the motion artifacts from signals generated by the SCG sensor are at least partially cancelled.

In some instances of the stacked-design multi-mode conformable epidermal sensor, one or more of the ECG sensor, the SCG sensor, the PPG sensor, and/or the temperature sensor may be electrically connected to one another and/or synchronized.

In some instances of the stacked-design multi-mode conformable epidermal sensor, the second flexible insulating substrate may be patterned in an island-serpentine design and the electronics may comprise islands of densely packed electronics connected by stretchable serpentine interconnects.

In some instances of the stacked-design multi-mode conformable epidermal sensor, the wireless communications interface may comprise a Bluetooth low-energy transceiver.

In some instances of the stacked-design multi-mode conformable epidermal sensor, the one or more biocompatible electrodes of the ECG sensor and the transparent first flexible, stretchable insulating substrate can be separated from the electronics and the second flexible insulating substrate and discarded such that the electronics and the second flexible insulating substrate can be reused to form a second multi-mode epidermal sensor.

In some instances the stacked-design multi-mode conformable epidermal sensor may be lightweight, having a weight of approximately 2.5 grams, or less. Advantageously, the light weight and conformal form factor of the stacked-design multi-mode conformable epidermal sensor leads to lower motion noise on the SCG and PPG during movement.

In some instances of the stacked-design multi-mode conformable epidermal sensor, the stacked-design multi-mode conformable epidermal sensor may be stretched in any direction up to approximately 20 percent over its normal size and maintain sensor accuracy.

In some instances of the stacked-design multimode sensor, the stacked-design multi-mode sensor has at any time total power consumption of approximately 7 milli-watts, or less.

In some instances of the stacked-design multi-mode sensor, the transparent first flexible, stretchable insulating substrate comprises a hydrocolloid medical dressing with adhesive on the first side to adhere to the epidermis.

In some instances of the stacked-design multi-mode sensor, a thin substrate made of a silicone elastomer, such as Polydimethylsiloxane (PDMS), or adhesive tape could be used to cover the electronic parts.

In some instances of the stacked-design multi-mode conformable epidermal sensor, the transparent first flexible, stretchable insulating substrate may have a thickness of less than approximately 50 microns.

In some instances of the stacked-design multi-mode conformable epidermal sensor, the first flexible, stretchable insulating substrate may have dimensions of approximately 65 millimeters by 40 millimeters.

In some instances of the stacked-design multi-mode conformable epidermal sensor, the electrode pattern on the first side of the transparent first flexible, stretchable insulating substrate may be serpentine shaped.

In some instances of the stacked-design multi-mode conformable epidermal sensor, each electrode pattern may include one or more terminal pads for connection to an interconnect.

In some instances, the stacked-design multi-mode sensor may further comprise a third flexible substrate that substantially or completely covers the first flexible, stretchable insulating substrate and the second flexible substrate.

In some instances, the stacked-design multi-mode sensor may further comprise motion artifact removal. For example, the motion artifact removal may comprise an implementation of adaptive filters and signals decomposition techniques, such as Empirical Mode Decomposition (EMD), in a sequential stack for motion compensation. In some instances, the motion compensation framework may comprise ensemble averaging multiple beats. In some instances, the implementation of adaptive filters may comprise one or more of Recursive Least Squares (RLS) filters, Least Mean Squares (LMS) filters, and Normalized Least Mean Squares (NLMS) filters.

In another aspect, a method for using a stacked-design multi-mode conformable epidermal sensor is described. One implementation of the method may comprise attaching a stacked-design multi-mode conformable epidermal sensor to an epidermis, proximate to the heart. The stacked-design multi-mode conformable epidermal sensor may comprise: a transparent first flexible, stretchable insulating substrate comprising a first side and a second side opposite the first side, wherein the first side adheres to an epidermis and the transparent first flexible, stretchable insulating substrate conforms to the epidermis; an electrocardiogram (ECG) sensor comprising one or more biocompatible electrodes, wherein each electrode forms an electrode pattern on the first side of the transparent first flexible, stretchable insulating substrate and each electrode is configured to flex and stretch with the transparent first flexible, stretchable insulating substrate to conform to the epidermis; a seismocardiogram (SCG) sensor, said SCG sensor mounted on a second side of a second flexible insulating substrate, said second flexible insulating substrate having a first side that is substantially in contact with the second side of the transparent first flexible, stretchable insulating substrate; and electronics disposed at least partially on the second side of the second flexible insulating substrate, said electronics comprising at least a wireless communications interface, a central processing unit, an accelerometer used by the SCG sensor, the ECG sensor, and a power source, wherein components of the electronics are separated from one another on the second side of the second flexible insulating substrate and connected to one another using copper traces configured to flex and stretch with the transparent first flexible, stretchable insulating substrate and/or the second flexible insulating substrate, and to conform to the epidermis, wherein the electronics are connected to the one or more biocompatible electrodes of the ECG sensor using conductive material such as anisotropic conductive film (ACF) windows in the transparent first flexible, stretchable insulating substrate, or conductive materials (e.g., ACF windows) in both the transparent first flexible, stretchable insulating substrate and the second flexible insulating substrate. ECG test equipment is connected to ECG sensor and SCG test equipment to the SCG sensor; and at least an electrocardiogram and seismocardiogram are simultaneously measured using the ECG test equipment and the SCG test equipment, respectively.

In some instances of the method, the stacked-design multi-mode conformable epidermal sensor may further comprise a photoplethysmogram (PPG) sensor disposed on the first side of the second flexible insulating substrate. The PPG sensor may comprise one or more light emitting diodes (LEDs) and one or more photodetectors (PDs) that perform reflective photoplethysmography through the transparent first flexible, stretchable insulating substrate. The one or more LEDs and PDs may be electrically connected to the electronics disposed at least partially on the second side of the second flexible insulating substrate through the second flexible insulating substrate.

In some instances of the method, the stacked-design multi-mode conformable epidermal sensor may further comprise a temperature sensor. The temperature sensor may be disposed on the first side of the second flexible insulating substrate and connected to the electronics disposed at least partially on the second side of the second flexible insulating substrate through the second flexible insulating substrate.

In some instances of the method, signals from one or more of the ECG sensor, the SCG sensor, the PPG sensor, and the temperature sensor of the stacked-design multi-mode conformable epidermal sensor are synchronized with the wireless communications interface and/or the central processing unit of the stacked-design multi-mode conformable epidermal sensor.

In some instances the method may further comprise computing a beat-to-beat blood pressure (BP) from the electrocardiogram, seismocardiogram and photoplethysmogram.

In some instances the method may further comprise measuring one or more cardiac time intervals (CTIs) including systolic timing intervals (STIs) using the stacked-design multi-mode conformable epidermal sensor. The STIs may include a pre-ejection period (PEP) and a left ventricular ejection time (LVET). The PEP and LVET may be used to assess left ventricular (LV) performance and to predict other cardiac indexes such as CO and/or SV.

In some instances of the method, motion artifacts from signals generated by the SCG sensor of the stacked-design multi-mode conformable epidermal sensor may be at least partially cancelled.

In some instances of the method, the second flexible insulating substrate may be patterned in an island-serpentine design and the electronics may comprise islands of densely packed electronics connected by stretchable serpentine interconnects.

In some instances of the method, the epidermis may be located on a chest of a human.

In some instances of the method, the stacked-design multi-mode conformable epidermal sensor is lightweight, weighing approximately 2.5 grams or less, and the light weight and conformal form factor of the stacked-design multi-mode conformable epidermal sensor may lead to lower motion noise on the SCG and PPG during movement.

In some instances the method may further comprise the use of motion artifact removal. In some instances, the motion artifact removal may comprise an implementation of adaptive filters and/or and/or ensemble averaging and/or signal decomposition techniques such as Empirical Mode Decomposition (EMD) in a sequential stack for motion compensation. In some instances, the implementation of adaptive filters may comprise one or more of Recursive Least Squares (RLS) filters, Least Mean Squares (LMS) filters, and Normalized Least Mean Squares (NLMS) filters.

In some instances of the method one or more features such as CTIs, and pulse transit time (PTT) may be extracted from the processed signals and used for BP estimation, and SV and CO prediction using additional techniques.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1A illustrates synchronized electrocardiogram (ECG) and seismocardiogram (SCG) providing a complementary view of cardiac activities, including the QRS complex and T-wave in ECG, and aortic valve opening (AO), aortic valve closing (AC), mitral valve closing (MC), and mitral valve opening (MO) in SCG. Pre-ejection period (PEP) is from the Q peak or alternatively R peak to the AO peak and left ventricular ejection time (LVET) is from the AO peak to the AC peak.

FIG. 1B is a schematic illustrating different phases in the left ventricular heart cycle, highlighting the sequential opening and closure of the mitral valve (MV) and aortic valve (AV), as well as cardiac time intervals (CTI) including iso-volumetric contraction time (IVCT), LVET, iso-volumetric relaxation time (IVRT), and ventricular filling time (VFT).

FIG. 2 illustrates an exemplary stacked-design multi-mode conformable epidermal sensor comprising an electrode layer, a flexible printed circuit (FPC) layer, and, typically, a cover layer.

FIG. 3 shows the synchronized ECG and SCG waveforms of an embodiment of the stacked-design multi-mode conformable epidermal sensor ensembled over five heartbeats to reduce noise with the relevant cardiac features (e.g., QRS complex, AO, AC) marked.

FIG. 4 illustrates a cross-section view of an exemplary stack-up of an embodiment of a stacked-design multi-mode conformable epidermal sensor.

FIG. 5 illustrates transferring the graphite film of the one or more electrodes onto a commercial medical dressing (e.g., Tegaderm™, 3M, St. Paul, MN).

FIGS. 6A and 6B illustrate two different instances of the disclosed stacked-design multi-mode conformable epidermal sensor showing that that the flexible, lightweight, and stretchable design allows it to conform to the macro contours of the human body for easy lamination on the human chest.

FIG. 7A is an illustration of an exemplary electronic subsystem of one embodiment of the disclosed stacked-design multi-mode conformable epidermal sensor.

FIG. 7B illustrates a sleep/wake process for the CPU of FIG. 7A, which helps to conserve energy resources for the stacked-design multi-mode conformable epidermal sensor.

FIG. 7C illustrates current draw of the exemplary stacked-design multi-mode conformable epidermal sensor shown in FIG. 7A.

FIG. 7D is an illustration of an exemplary electronic subsystem of an alternate embodiment of the disclosed stacked-design multi-mode conformable epidermal sensor.

FIGS. 8A-8C illustrate that serpentine interconnects between different groups of electronics allow the disclosed stacked-design multi-mode conformable epidermal sensor to stretch reliably up to 20% strain (applied uni-axially) without any damage or drop in signal quality.

FIGS. 9A-9D illustrate the easy re-usability of the disclosed stacked-design multi-mode conformable epidermal sensor.

FIG. 10A shows the ECG signals from both electrodes comprised of dry graphite film and conventional gel electrodes, temporarily connected to the ECG sensor of the e-tattoo, have a similar signal-to-noise ratio (SNR) and a very well-defined QRS complex.

FIG. 10B shows the SCG signal collected with the disclosed stacked-design multi-mode conformable epidermal sensor compared against an M-mode echocardiogram.

FIG. 10C illustrates the ability of the disclosed stacked-design multi-mode conformable epidermal sensor to determine a rough orientation of the user by analyzing the low-frequency components.

FIG. 10D illustrates testing the performance of the SCG sensor of the disclosed stacked-design multi-mode conformable epidermal sensor, where a synthetic voltage and vibration-generating phantom for replicating human-like ECG and SCG signals was used and the quality of collected SCG signals against the known signal input to the phantom is evaluated.

FIG. 10E illustrates that real human SCG signals were fed into the generator and were accurately reproduced by the disclosed stacked-design multi-mode conformable epidermal sensor.

FIG. 11A illustrates a block diagram of a pre-processing pipeline designed for extracting SCG features in an exemplary stacked-design multi-mode conformable epidermal sensor.

FIGS. 11B and 11C illustrate AO and AC time locations extracted using an SCG peak tracking algorithm.

FIG. 12A illustrates a set of experiments, conducted on five participants, comprised of holding static poses and performing cycling under incremental load with breaks.

FIG. 12B is a Bland Altman plot comparing heart rate (HR) from the disclosed stacked-design multi-mode conformable epidermal sensor and a Non-invasive Cardiac Output Monitor (NICOM) during the static poses experiment, which shows a strong agreement for all participants with a difference of 0.07±1.21 beats per minute (bpm).

FIGS. 12C and 12D show the change in average PEP and LVET respectively, measured in milliseconds (ms), for different participants as they transitioned from lying supine to sitting upright and then to standing.

FIG. 12E shows the HR from the disclosed stacked-design multi-mode conformable epidermal sensor and from the NICOM during the cycling exercise experiment.

FIG. 12F shows the Bland-Altman plot comparing the LVET from the disclosed stacked-design multi-mode conformable epidermal sensor and NICOM for each participant after the entire cycling experiment.

FIG. 12G shows the correlation plot between the LVET measurements from the disclosed stacked-design multi-mode conformable epidermal sensor and the NICOM with a Pearson correlation coefficient of 0.955.

FIG. 13A is an illustration of the FAD framework for both electrocardiogram (ECG) and seismocardiogram (SCG) signals, from bandpass filtering (BPF) to adaptive normalized least mean square (NLMS) filtering, subsequent multiple beat ensemble averaging, and finally applying empirical mode decomposition (EMD) to recover a clean SCG signal. Extraction of the key cardiac time intervals of interest, namely pre-ejection period (PEP) and left ventricular ejection time (LVET) is performed on this recovered signal in conjunction with the processed ECG signal.

FIG. 13B illustrates results achieved through the FAD framework aimed at motion compensation on SCG signals during walking. 3 seconds of raw signal, along with a recovered heartbeat from the SCG signal exclusively is illustrated. Notably, the recovered waveform exhibits a quality comparable to that of the signal at rest.

FIG. 13C illustrates results achieved through the FAD framework aimed at motion compensation on SCG signals during cycling. 3 seconds of raw signal, along with a recovered heartbeat from the SCG signal exclusively is illustrated. Notably, the recovered waveform exhibits a quality comparable to that of the signal at rest.

FIG. 14A illustrates heart-rate comparisons between embodiments of the disclosed stacked-design multi-mode conformable epidermal sensor and a wearable smartwatch.

FIGS. 14B-14D illustrate that during restful segments of long-term wear of the disclosed stacked-design multi-mode conformable epidermal sensor that the ECG and SCG data is mostly devoid of any motion artifacts.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

The ventricular function of a heart can be divided into four phases: isovolumic relaxation, ventricular filling, isovolumic contraction, and rapid ventricular ejection. Isovolumetric relaxation and ventricular filling constitute the ventricular diastolic phase. During the ventricular filling the mitral valve is open, and blood is flowing into the ventricles. After the ventricle fills and transitions to contracting, the pressure eventually exceeds that of the atrium. This gradient closes the mitral valve, which marks the beginning of systole. The time between the closing of the mitral valve and the subsequent opening of the aortic valve is the isovolumic contraction (IVC) period. The aortic valve opens when the pressure within the ventricle exceeds the pressure in the aorta, marking the beginning of rapid ventricular ejection. After the aortic valve closes, the cycle begins again. Impairment of the heart's pumping function, known as systolic congestive heart failure, or heart failure with reduced ejection fraction, can prevent the proper flow of oxygen and nutrients to target organs. Parameters such as stroke volume (SV), cardiac output (CO), and ejection fraction (EF) provide important insights into the health and performance of the heart.

However, a lack of accurate, non-invasive, portable, and/or easy-to-use devices for measuring these parameters hinders their widespread adoption for cardiovascular health assessment. Cardiac Time Intervals (CTI), comprising Systolic timing intervals (STI), which measure the duration of cardiac phases, have been demonstrated as viable alternatives to these parameters for assessing cardiac performance. The pre-ejection period (PEP) is the time between the electrical excitation of the ventricular cardiomyocytes and the opening of the aortic valve (AO). This includes the electromechanical delay from the electrical depolarization of the ventricles to the start of ventricular contraction as well as the IVC time. PEP provides insight into myocardial contractility as an increase in contractility results in a sharper increase in left ventricular (LV) pressure thereby shortening the PEP. The left ventricular ejection time (LVET) is the time from AO to the closure of the aortic valve (AC) and represents the total time blood flows from the ventricle into the aorta. The sum of these two periods (electromechanical systole or QS₂) is the total time the heart spends in ventricular systole.

The PEP and L VET offer a convenient non-invasive method to assess LV performance. People suffering from heart failure exhibit a lengthening of PEP and shortening of LVET in resting conditions mostly due to a diminished rate of isovolumetric pressure rise, and an inability to maintain a high LV pressure during the ejection period associated with decreased cardiac contractility, respectively. As a result, the ratio between PEP and LVET may be used as a good indicator of LV dysfunction. Although common, solely using an electrocardiogram (ECG) to track cardiac health is limited as it can only sense the electrical activity of the heart and is unable to provide any information about the mechanical activity of the heart such as contraction and valve activity. Indeed, even though the electrical stimulus for contraction is intact, the actual contractile force of the myocardial cells may be altered. Several non-invasive methods for capturing these mechanical cardiac events have been introduced. Impedance cardiography (ICG) devices measure the impedance change caused by blood being pumped by the heart, and the features from the corresponding impedance waveform can be related to different cardiac events, such as AO. In healthy subjects with low body mass index (BMI), this technique can provide accurate measures of STIs; however, in broader populations with cardiovascular diseases and in patients with higher BMI, ICG has been shown to have high error compared to reference standard invasive measures. Additionally, ICG requires electrodes spaced across the whole thorax, requiring trained personnel for positioning, and making unobstructed monitoring very difficult. Phonocardiograms (PCG) capture the acoustic signals generated by the heart valves. However, cardiac acoustic signals are only generated by the closure of the valves and thus cannot provide any indication of the opening of the aortic valve as needed for both PEP and LVET. More recently, ballistocardiogram (BCG) and seismocardiogram (SCG) measurements have become popular for mechanical heart function assessments. The BCG signal is caused by the motion of the whole body generated by the ejection of blood by the heart and the movement of blood through the vasculature but requires special scales or beds to capture. The SCG signal is a more localized measurement of the chest vibrations associated with cardiac contraction and blood movement, usually measured at or near the sternum. While similar to PCG, SCG senses in a much lower frequency range (up to 40 Hz) compared to PCG (up to 750 Hz).

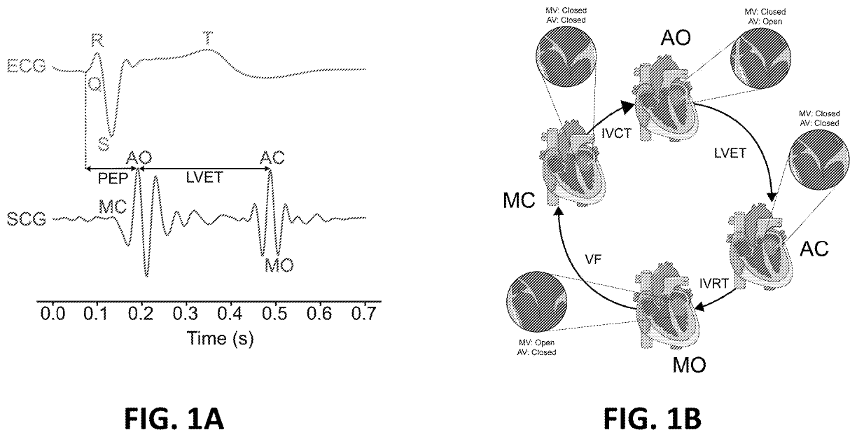

SCG's accuracy in detecting the precise moments of cardiac valve movements is known. FIG. 1A displays synchronized ECG and SCG signals for a single heartbeat, with key features clearly labeled. FIG. 1B shows diagrams that highlight important valve movements and their related timings within a cardiac cycle. The start of ventricular systole, indicated by the first peak in SCG right after the R peak in ECG, precisely captures the closure of the mitral valve (MC). It is immediately followed by the first major peak in SCG, known as the aortic valve opening (AO) peak, which marks the beginning of the ejection period. The second major peak in SCG records the aortic valve closing (AC), which is immediately followed by the mitral valve opening (MO) peak, which signify the start of diastole and the initiation of ventricular filling, respectively. The precise timing information enables the calculation of key CTIs, such as the pre-ejection period (PEP) and the left ventricular ejection time (LVET), which are vital for assessing heart function. For example, it is known that PEP and LVET have correlation with cardiac ejection fraction (EF) and left ventricular failure.

However, the development of wearable SCG sensors for everyday use has been hindered by motion artifacts. The motion sensors used to collect SCG from the surface of human chest inadvertently capture all body movements including respiration, not solely heart valve actions, leading to considerable signal contamination. The issue of motion artifacts is evident when noting that the sternal vibrations due to the heartbeat induce accelerations typically between 5-10 milli-g (mg) (1 g=9.8 m/s2), in contrast to activities of daily living like walking, which can produce accelerations of several hundreds of mg. Additionally, the noise from motion artifacts overlaps with the frequencies of the SCG signal, which typically range from 1-40 Hz, depending on the phenomena being studied. The primary heartbeat component is generally found within the 0.5-2 Hz range, while additional components and harmonics may reach higher frequencies. Frequencies associated with daily activities also fall within this low-frequency range. The considerable difference in magnitudes and the overlapping frequency bands cause the difficulty in isolating and precisely interpreting SCG signals during movements. Addressing this challenge is desired for advancing the viability of ambulatory SCG monitoring in real-life scenarios.

Disclosed herein are embodiments of a thin, macro-conformal, stretchable, and lightweight sensing platform that can be laminated onto the human chest like a temporary tattoo and hence termed an electronic- or e-tattoo. The e-tattoo uses multi-mode electro-mechanical sensing by utilizing ECG and SCG sensors. The ECG sensor interfaces with the human body via biocompatible electrodes (for example, graphite film electrodes though other biocompatible electrode material is contemplated within the scope of this disclosure) and the SCG is measured using a high-resolution, low-noise accelerometer that can detect subtle vibrations caused by the heart. Wireless connectivity incorporating, for example, Bluetooth Low Energy (BLE) is utilized to stream the data in real-time to a host device. External synchronization between the ECG and SCG ensures high cross-modality timing accuracy. Synchronization also allows reduced power consumption when compared to conventional devices. In some embodiments, the e-tattoo further comprises optical (photoplethysmogram (PPG)) and/or thermal (temperature) sensing. The e-tattoo has a stacked-design, which facilitates conformability, stretchability and easy fabrication/reuse.

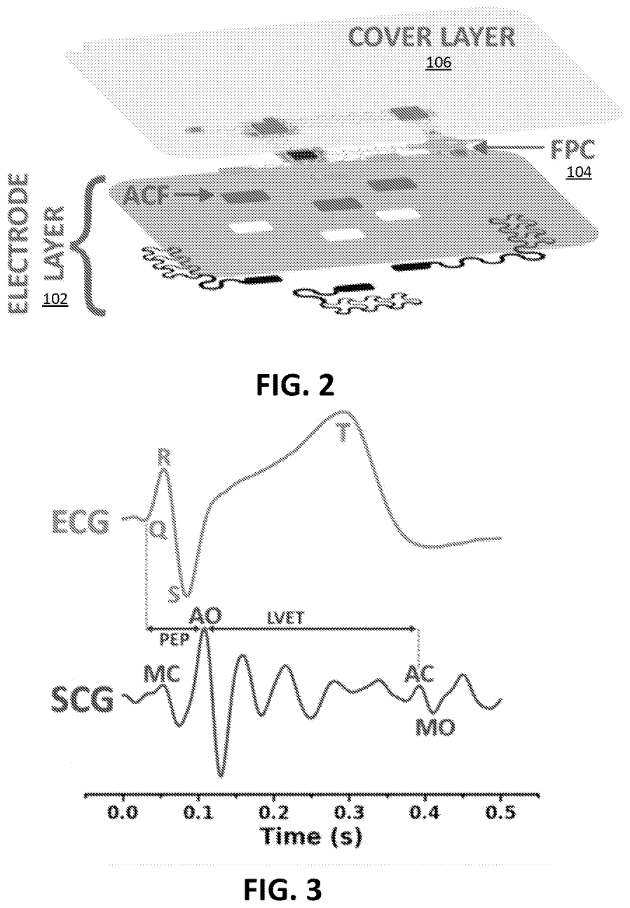

Generally, as shown in FIG. 2, embodiments of the device comprise an electrode layer 102, a flexible printed circuit (FPC) layer 104, and typically, a cover layer 106.

The electrode layer 102 comprises one or more electrodes that are made from biocompatible materials such as graphite polyurethane film and laminated onto a first side of a transparent first flexible, stretchable insulating substrate. Each electrode forms an electrode pattern on the first side of the transparent first flexible, stretchable insulating substrate and each electrode is configured to flex and stretch with the transparent first flexible, stretchable insulating substrate to conform to the epidermis of a wearer. In some instances, the electrode pattern on the first side of the transparent first flexible, stretchable insulating substrate is serpentine shaped, and each electrode pattern generally includes one or more terminal pads for connection to an interconnect.

In some instances, the transparent first flexible, stretchable insulating substrate comprises a polyurethane film medical dressing, such as Tegaderm™ (3M, Saint Paul, MN), having an adhesive layer on the first side of the transparent first flexible, stretchable insulating substrate. Electrical contact is made with the electrodes from a second side of the transparent first flexible, stretchable insulating substrate through holes defined by the transparent first flexible, stretchable insulating substrate and a conductive material such as an anisotropic conductive film (ACF) acts as an adhesive and conductor between electrodes and electronics. As stable electrode contact resistance is desired for good ECG signal quality, this design has shown that stretching the serpentine electrode design showed negligible resistance change up to 40% strain.

Further comprising embodiments of the device is the flexible printed circuit (FPC) layer 104. In some instances, the FPC layer 104 may be covered with a third flexible substrate 106 that covers the first flexible, stretchable insulating substrate and the second flexible substrate. In some instances, this third layer of material 106 may comprise Tegaderm™. In some instances, a portion of the third flexible substrate 106 may be removed to define one or more holes that expose a power source (e.g., a battery) mounted on the FPC layer. In this way, the power source can be replaced as needed without having to replace the entire device.

Generally, the FPC layer 104 comprises electronics disposed at least partially on a second side of the second flexible insulating substrate. In some instances, the second flexible insulating layer comprises polyimide. Typically, the first side of the second flexible insulating substrate is in substantial contact with the second side of the first flexible, stretchable insulating substrate. Generally, the electronics comprise at least a wireless communications interface, a central processing unit, an accelerometer used by the SCG sensor, an ECG sensor, and a power source. Components of the electronics are separated from one another on the second side of the second flexible insulating substrate and connected to one another using conductors configured to flex and stretch with the transparent first flexible, stretchable insulating substrate and/or the second flexible insulating substrate, and to conform to the epidermis. For example, the conductors may comprise copper or gold traces having a serpentine pattern. As noted herein, the electronics are connected to the one or more biocompatible electrodes of the ECG sensor using conductive material such as anisotropic conductive film (ACF) windows in the transparent first flexible, stretchable insulating substrate, or ACF windows in both the transparent first flexible, stretchable insulating substrate and the second flexible insulating substrate.

In other embodiments of the device, the electronics may further comprise a photoplethysmogram (PPG) sensor disposed on the first side of the second flexible insulating substrate. Generally, the PPG sensor comprises one or more light emitting diodes (LEDs) and one or more photodetectors (PDs) that perform reflective photoplethysmography through the transparent first flexible, stretchable insulating substrate. The one or more LEDs and PDs are electrically connected to the electronics disposed at least partially on the second side of the second flexible insulating substrate through the second flexible insulating substrate.

In yet other embodiments of the device, the electronics may further comprise a temperature sensor. The temperature sensor is disposed on the first side of the second flexible insulating substrate and connected to the electronics disposed at least partially on the second side of the second flexible insulating substrate through the second flexible insulating substrate.

In some instances, the electronics may further comprise a second accelerometer disposed on the second side of the second flexible insulating substrate. The second accelerometer may be used at least for motion compensation and cancellation of motion artifacts. The second accelerometer may also be used for body orientation detection and activity recognition.

In some instances, one or more of the ECG sensor, the SCG sensor, the PPG sensor, and the temperature sensor are electrically connected to one another for synchronization purposes. For example, one or more of the ECG sensor, the SCG sensor, the PPG sensor, and the temperature sensor may be connected using the conductors configured to flex and stretch with the transparent first flexible, stretchable insulating substrate. Synchronization of one or more of the ECG sensor, the SCG sensor, the PPG sensor, and the temperature sensor with the wireless communications interface and/or a central processing unit or using a peripheral synchronization that excludes the CPU and thus lowers poser consumption of the device, thus increasing the life of the power source enabling long term measurements. Furthermore, synchronization of the components reduces timing noise artifacts. FIG. 3 shows the synchronized ECG and SCG waveforms ensembled over five heartbeats to reduce noise with the relevant cardiac features (e.g., QRS complex, AO, AC) marked.

Together, the electronics and the second flexible insulating substrate comprise the FPC layer 104. It should be noted that the electronics are completely isolated from contact with the human body (i.e., epidermis) and the only components in direct skin contact are the biocompatible electrodes and the first flexible, stretchable insulating substrate.

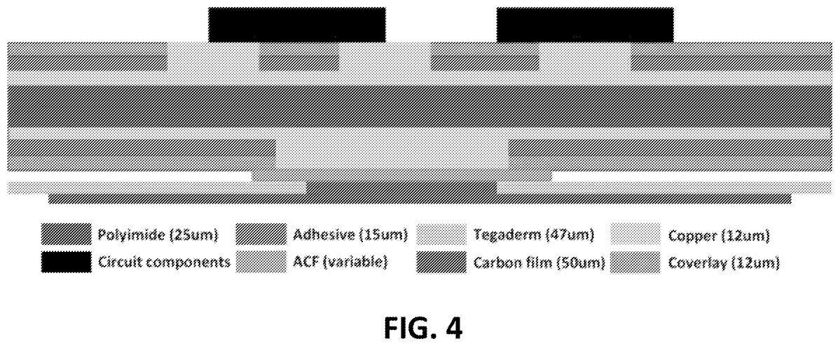

In one exemplary embodiment the FPC layer 104 comprises a double-layer flexible printed circuit board with polyimide (PI) as the substrate and copper traces on top and bottom. It is encapsulated with additional PI layers on both sides to prevent unintentional electrical contact. The exposed copper for connecting to the integrated circuits (IC) and other circuit components undergoes an Electroless Nickel Immersion Gold (ENIG) surface plating process. The total thickness of the FPC is around 100 μm, and a more detailed exemplary stack-up of a stacked-design multi-mode conformable epidermal sensor is shown in FIG. 4. The circuit components are soldered onto the FPC with lead (Pb) free soldering paste to make it safer for use on human subjects. The FPC is patterned in an island-serpentine design comprising islands of densely packed electronics connected by stretchable serpentine interconnects. The electrode for the ECG is made with biocompatible materials such as, for example, graphite film (graphite polyurethane film, Mineral Seal Corporation, Tucson, AZ), which has been laser cut into serpentine-like patterns to make it stretchable.

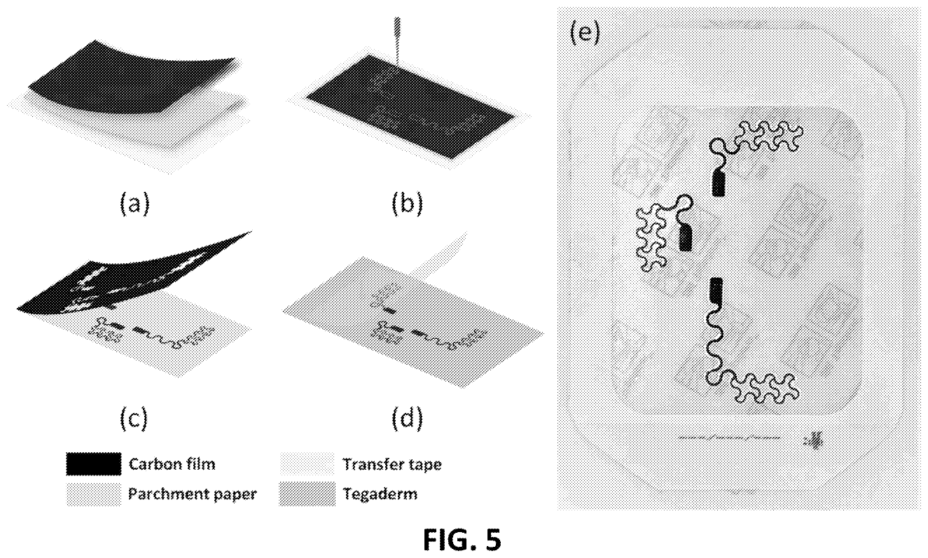

Using the “cut-and-paste” subtractive manufacturing process (see S. Yang, Y.-C. Chen, L. Nicolini, P. Pasupathy, J. Sacks, B. Su, R. Yang, D. Sanchez, Y.-F. Chang, P. Wang, et al., Advanced Materials 2015, 27, 41 6423, which is fully incorporated by reference), the graphite film is transferred onto a commercial medical dressing (e.g., Tegaderm™, 3M) as is illustrated in FIG. 5. The exemplary graphite film dry electrode manufacturing process shown in FIG. 5 includes a) laminating carbon film on parchment paper and transfer tape, b) laser cutting serpentine electrode pattern, c) removing excess carbon film, d) transferring to Tegaderm™, and e), a ready-to-use electrode transferred onto a Tegaderm™ patch. Without active circuit components, the total thickness of the FPC and electrode layer is only about 200 μm. Small holes are cut in the medical dressing to expose a part of the electrodes where a piece of conductive material such as anisotropic conductive film (ACF) is attached. The ACF material is epoxy/acrylate-blend adhesive system filled with 43-micron silver-coated glass beads (Anisotropic Conductive Film 7303, 3M, St. Paul, MN). After the electronics are mounted over the ACF, pressure is applied to activate the ACF and form an electrical connection between the electrodes and the ECG front end.

The flexible, lightweight, and stretchable design of the disclosed device allows it to conform to the macro contours of the human body for easy lamination on the human chest as shown in FIGS. 5A and 5B. This form factor coupled with firmware for sensor interfacing and transmission of data allows achievement of one of the lowest total device weights (2.5 g including battery) and current draws (940 uA) among wearable cardiac sensors. This low weight makes the device comfortable to wear and also reduces motion artifacts when the user is moving. The ECG, SCG, PPG, and other acquired data (e.g., temperature, packet count) is transmitted in real-time over the communications interface (e.g., BLE) to a host device such as a mobile phone, laptop computer, router, and the like. In some instances, software running on the host device may be used to connect to the e-tattoo device and receive and store the streamed data for later use. The application also provides user interfaces for visualizing the data in real-time for verifying the signal quality and marking important events. Markers that are placed using this interface are parsed as labels at specific timestamps in post-processing, allowing the user to annotate the data properly.

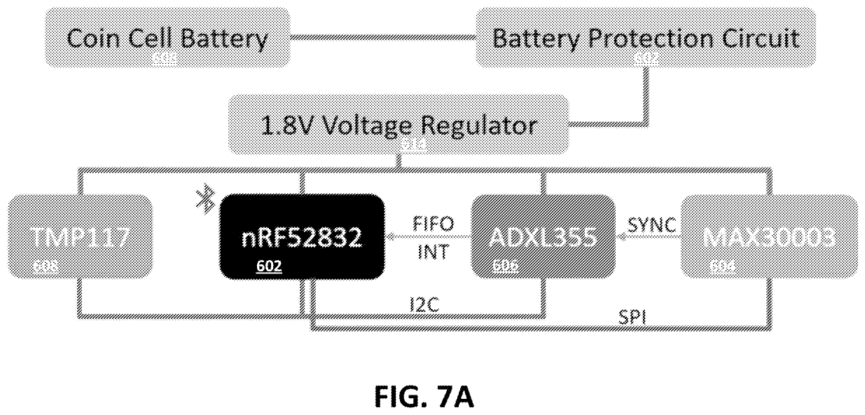

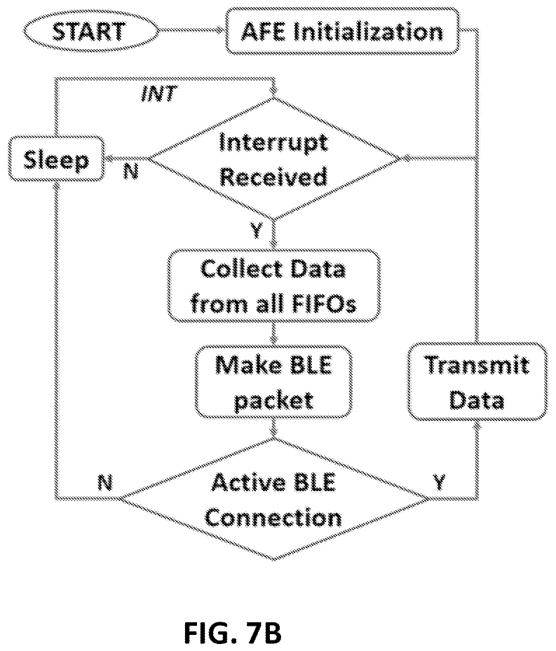

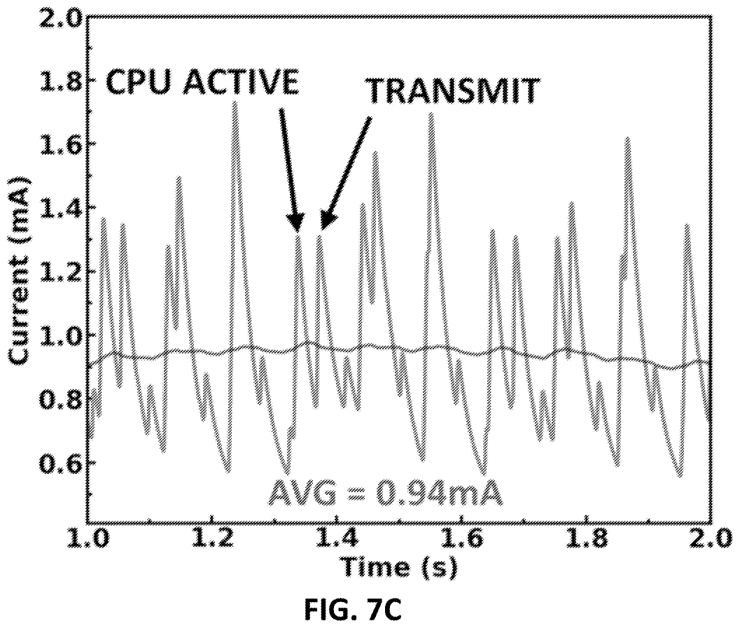

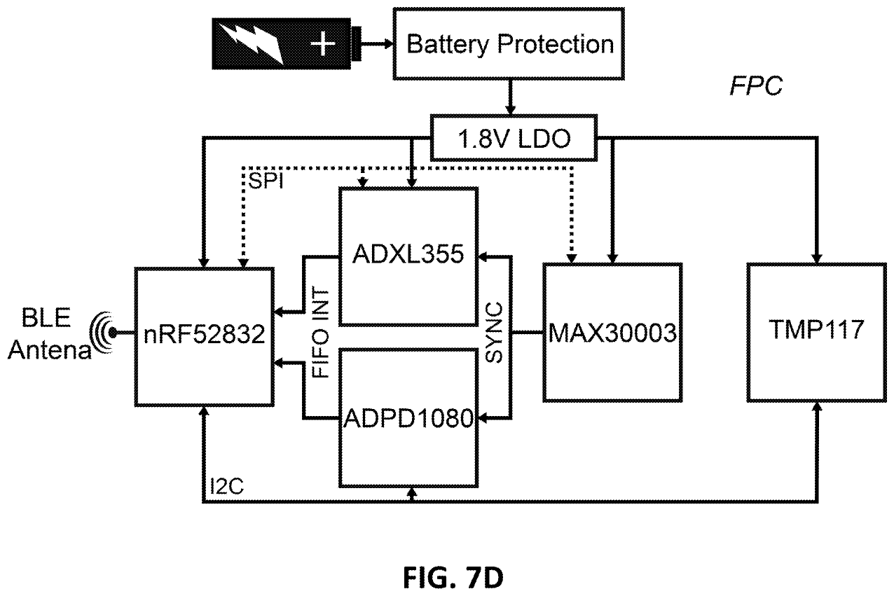

FIG. 7A is an illustration of an exemplary electronic subsystem of one embodiment of the disclosed stacked-design multi-mode conformable epidermal sensor. The stacked-design multi-mode conformable epidermal sensor shown in FIG. 7A comprises a central processing unit (CPU) with BLE capabilities 602. For example, the CPU 602 may comprise the nRF52832 from Nordic semiconductor for its ultra-low power consumption and sufficient processing power to perform required firmware tasks. The sensors are connected via different buses to the CPU 602. For example, the ECG sensor 604 may comprise a front end (MAX30003, Maxim Integrated), which is a single-lead ECG sensor with a right leg drive connected to the CPU 602 over a Serial Peripheral Interface (SPI). The ECG sensor 604 contains analog high-pass and low-pass filters. The high pass filter (cutoff frequency=0.5 Hz) filters out any DC drift in the ECG signal. The low pass filter (cutoff frequency=40 Hz) performs anti-aliasing and high frequency noise reduction. The SCG sensor 606, for example an ADXL355, Analog Devices, comprises a high-resolution, low-noise, MEMS-based accelerometer connected to the CPU 602 over the Inter-Integrated Circuit (I2C) interface. Both these ICs are completely self-sufficient (i.e., integrated analog front end, analog-to-digital converter, and digital readout), thus requiring minimal external components which reduces external noise contamination and power consumption. A digital temperature sensor 608 (for example a TMP117, Texas Instruments) sharing the I2C bus is located on the bottom (first) side of the FPC layer 104 allowing for more direct measurement of the skin. A PPG sensor, for example an ADPD1080, Analog Devices, may be situated on the second side of the FPC layer with one or more LEDs and photodiodes on the first side of the FPC layer to enable reflectance photoplethysmography though the transparent layer.

To achieve high accuracy in detecting CTIs the sampling interval must be free of variability. Furthermore, the synchronization between the ECG 604 and SCG 606 (and PPG) sensors also has to be very accurate, i.e., clock jitter and clock drift have to be kept at a minimum. This is especially true when trying to extract the PEP as it requires signals from separate sensors (i.e., ECG and SCG) and is much more sensitive to measurement error due to its relatively short absolute length (~100 ms). The disclosed data acquisition scheme achieves sub-ms-level synchronization while keeping power consumption to a minimum. To reduce clock jitter, an external 32.768 KHz crystal (KX201, Diodes Incorporated) with superb frequency stability (25 PPM) over a wide range of operating conditions can be used to generate the sampling clock for the ECG sensor. To prevent clock drift between the ECG 604 and SCG 606, a hardware synchronization method is used where the ECG sensor 604 generates a sync pulse on every sample. The SCG sensor 606 uses this pulse as an external sampling trigger. This ensures the samples on both sensors occur at the same time with a small, fixed, and deterministic delay. Both the sensors have internal queues (FIFOs) for storing data and this synchronization method makes it possible for the sensors to autonomously sample and store data locally without intervention from the CPU which can remain in a low-power sleep state. Once the FIFOs are full, an interrupt signal is sent to the CPU 602 which wakes up to collect the stored data from both sensors, compress it, and transmit it to a host device. This process is illustrated in FIG. 7B. This can be extended to the PPG sensor similarly to the SCG sensor. The ECG 604 and SCG sensor 606 is sampled at 125 Hz, and the FIFO size is kept at 20 samples, which translates to a CPU 602 wake frequency of 6.25 Hz. Because the CPU 602, the most power-hungry component, is kept in sleep mode most of the time, the average current draw of the exemplary stacked-design multi-mode conformable epidermal sensor while actively transmitting data is less than 1 mA with occasional spikes up to 2 mA when the CPU 602 becomes active and/or data is transmitted over BLE as shown in the current draw graph in FIG. 7C. Power is supplied to the stacked-design multi-mode conformable epidermal sensor from, for example, a small form factor coin cell battery (CR1220) 610 which is capable of providing power for up to 40 hours of operation. Analog filters 612 smooth out any potential current spikes from damaging the battery and a voltage regulator 614, for example a low drop-out (LDO) voltage regulator (NCP170, Onsemi) ensures proper voltage is supplied to the circuit components. In another implementation, an efficient buck converter can be used in place of the LDO.

FIG. 7D is an illustration of an exemplary electronic subsystem of an alternate embodiment of the disclosed stacked-design multi-mode conformable epidermal sensor. This embodiment of the device facilitates synchronized measurement of ECG (MAX30003), SCG (ADXL355), and PPG (ADPD1080) signals, along with intermittent measurement of skin temperature (TMP117). Each sensor is equipped with integrated analog front ends, ensuring that only digital data is communicated to the central processor. This design minimizes susceptibility to noise from external factors such as electromagnetic interference or body-coupled interference. To enhance signal fidelity, physiological measurements are synchronized using a peripheral-to-peripheral hardware synchronization scheme, effectively eliminating cross-modality timing errors. The central processing unit (nRF52832) is equipped with integrated Bluetooth Low Energy (BLE) capabilities, facilitating real-time transmission of data from the device to a designated receiver such as, for example purposes only, a smartphone running a custom-designed application. The smartphone accompanying application allows users to observe the signal waveform in real-time, ensuring the proper functionality of the device. Additionally, users can place markers at specific instances to mark significant events, providing a reference for post-processing analysis.

The ECG sensor necessitates robust electrical contact with the skin, a requirement fulfilled through the utilization of a biocompatible electrode material such as graphite polyurethane film laser cut in serpentine patterns and transferred onto a medical dressing (Tegaderm, 3M). Dry electrodes offer numerous advantages over conventional gel electrodes, such as immunity from signal degradation induced by electrode dehydration and ultra-thin form factor. To establish electrical connection between the layers, holes are punctured in the Tegaderm, and conductive material (e.g., z-axis conductive tape) is employed to link with the electrodes. This construction method creates a temporary connection between the device and the electrodes, allowing for the convenient disposal of electrodes post-use while facilitating the recycling of electronic components and FPC. It is important to note that, in this setup, only the medical dressing and biocompatible electrode material come in direct contact with the skin, ensuring isolation from the electronics. This design prioritizes user comfort, device re-usability, and long-term operation.

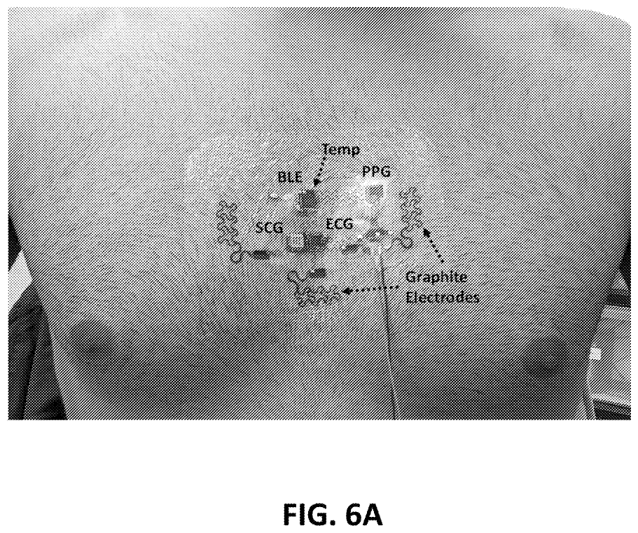

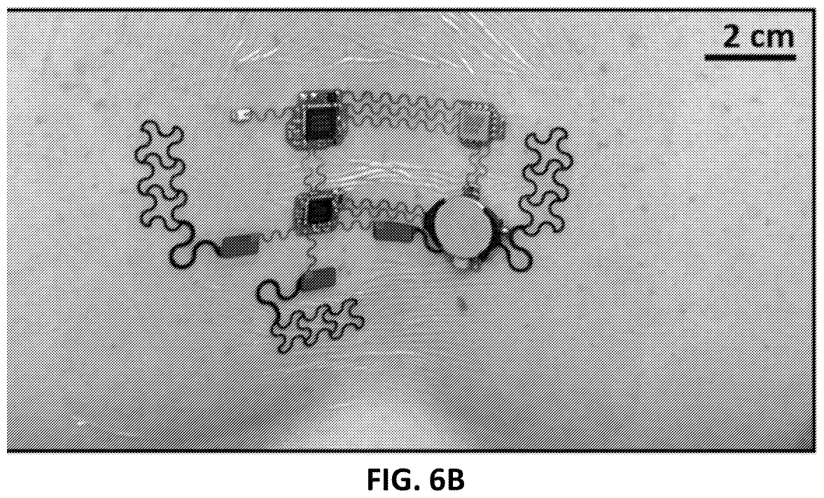

Generally, as shown in FIGS. 6A and 6B, embodiments of the device are laminated onto the chest such that the SCG sensor is placed in close proximity to the xiphoid process. The ECG electrodes span across the sternum and provide a strong signal with a sharp R-peak. However, within the realm of SCG, an ongoing challenge revolves around determining the optimal position for signal measurement. Currently, there is no standardized protocol for sensor placement for SCG recording. Areas such as the xiphoid process, then, are ideal for acquiring proper SCG signal morphology due to their minimal fat content and bony underlying support.

However, in several previous studies utilizing wearable SCG sensing systems during motion, applying the sensor near the xiphoid process was not entirely feasible due to the size, bulkiness, and rigidity of the hardware, leading to difficulties conforming to the significant curvature of the sternal crest. Research utilizing such devices near the xiphoid has typically necessitated the use of harnesses or belt-like apparatuses to secure the device in place. In contrast, many other investigations have opted for placement on the upper sternal region, closer to the suprasternal notch. However, this location has been deemed suboptimal for SCG sensing. The disclosed thin, flexible, and stretchable device presents a distinctive advantage by enabling application at the ideal position without concerns of delamination or compromising user comfort. This design choice addresses the limitations associated with traditional rigid systems, allowing for optimal SCG sensing in a challenging anatomical region.

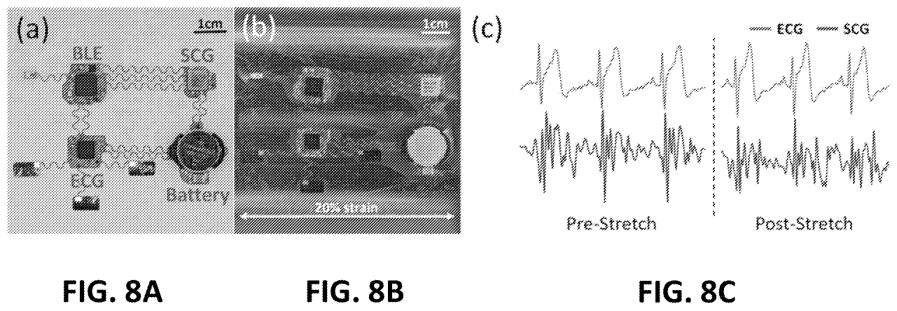

To be comfortable to wear, the disclosed stacked-design multi-mode conformable epidermal sensor conforms to the macro contours of the body, specifically the sternal region of the chest. It also is configured to stretch along with the skin when the user changes their posture or is in motion. Serpentine interconnects between different groups of electronics are used to enable the disclosed stacked-design multi-mode conformable epidermal sensor to maintain sensor accuracy by stretching reliably up to 20% strain (applied uni-axially) without any damage or drop in signal quality (see FIGS. 8A-8C). It should be noted that for obtaining the post-stretch data shown in FIG. 8C, the e-tattoo was relaxed and then reapplied on the human subject. Data collected from human subjects also indicate the maximum tensile strain of the central chest region is less than 20%. The disclosed stacked-design multi-mode conformable epidermal sensor also functions well under compressive strain and no delamination of electrodes was observed.

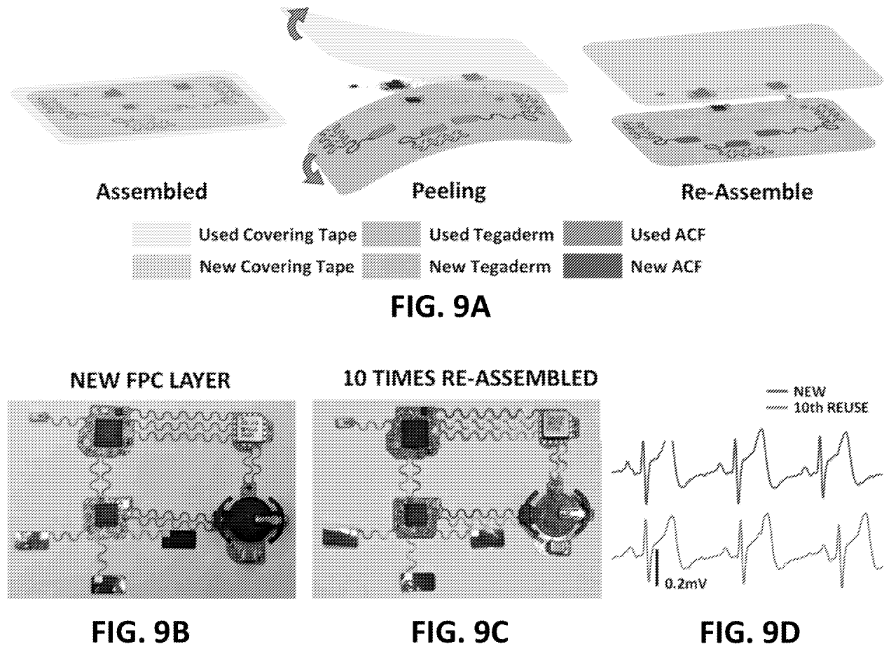

FIGS. 9A-9D illustrate the easy re-usability of the disclosed stacked-design multi-mode conformable epidermal sensor. The covering tape and electrode layer along with the conductive material (e.g., ACF window) can be peeled away and a new electrode layer can be attached. The electronics can be re-used multiple times with new electrode layers. This reduces the operational cost of the device as the electrode layer and covering tape is much cheaper than the electronics. Multiple peeling cycles do not damage the electronics significantly and there is no perceptible damage observed after reusing 10 times (FIGS. 9B, 9C). The ECG signal from a new stacked-design multi-mode conformable epidermal sensor and 10 times reused one also shows no major differences in signal quality (see FIG. 9D).

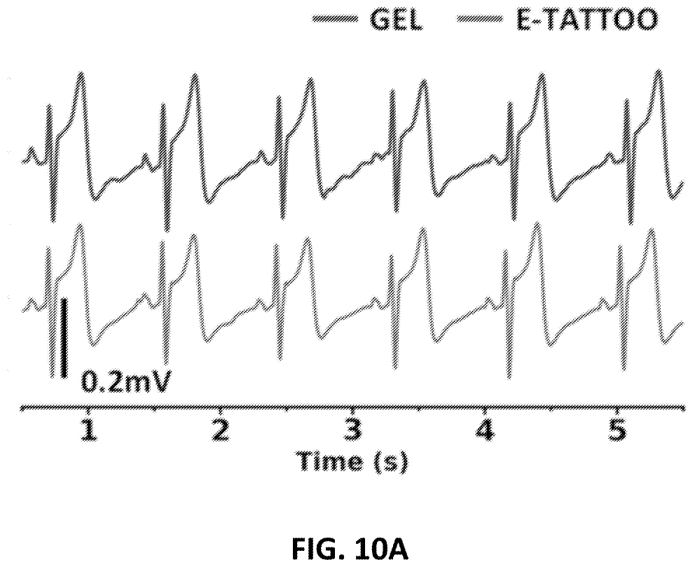

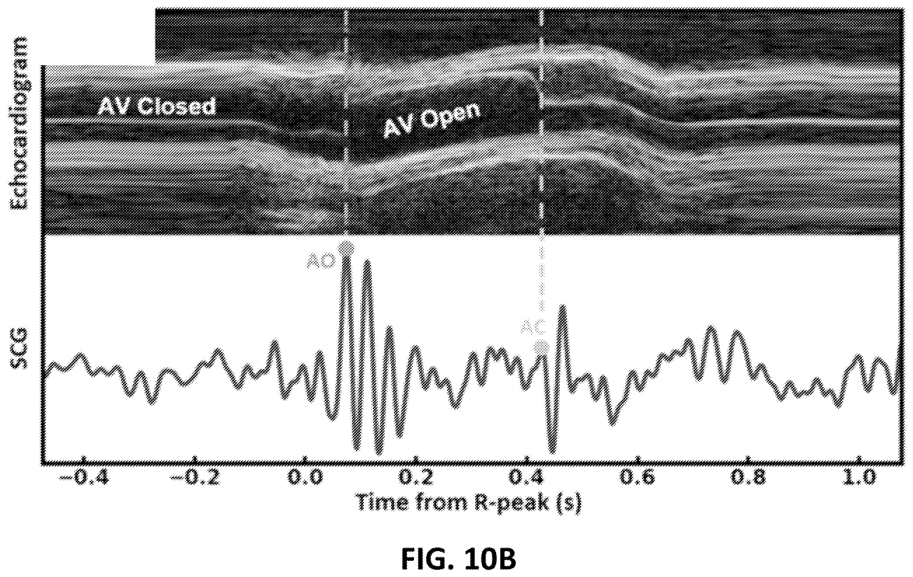

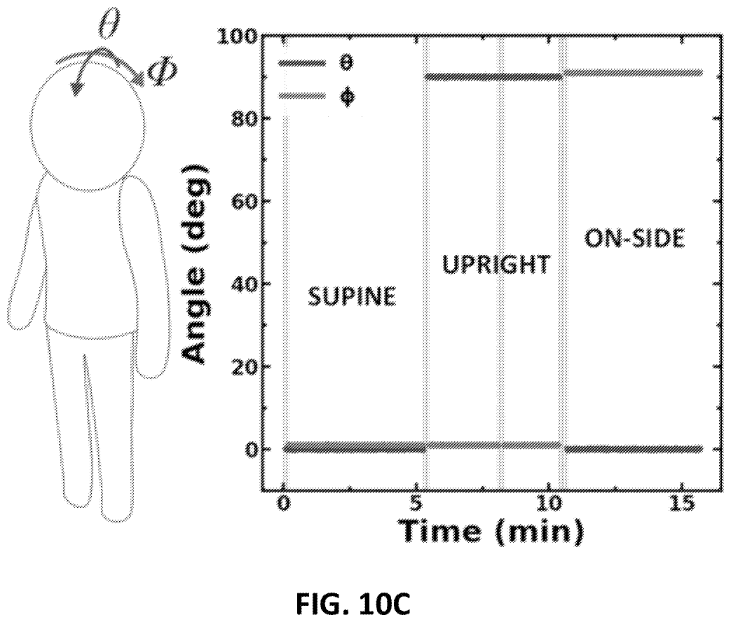

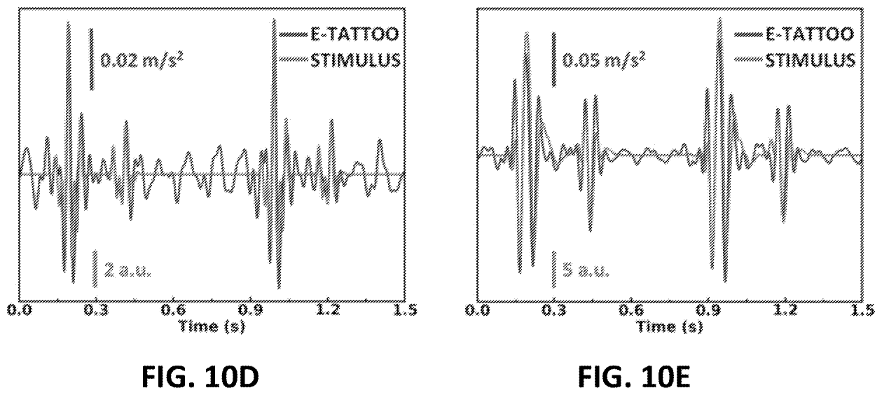

As a validation of the acquired disclosed stacked-design multi-mode conformable epidermal sensor signals, data from the ECG and SCG sensors were compared against data from standard clinical medical instruments. Firstly, the ECG signal acquired from both graphite film dry electrodes and commercial Ag/AgCl-based gel electrodes were compared. The centroid location of both types of electrodes was kept the same to minimize the effects of electrode position. FIG. 10A shows the ECG signals from both electrodes have a similar signal-to-noise ratio (SNR) and a very well-defined QRS complex. The elevated T-wave is a result of the short inter-electrode distance and not an issue caused by the dry electrodes themselves, evident both by the waveform acquired from the gel electrodes placed in the same location and in the literature. Dry electrodes are used as the disclosed stacked-design multi-mode conformable epidermal sensor may be applied on users for an extended duration of time. Previous work has shown the disadvantage of using gel electrodes for long durations. It should also be noted that as the gel dries during extended use the electrode contact impedance rises sharply, which makes the signal susceptible to external noise. FIG. 10B shows the SCG signal collected with the disclosed stacked-design multi-mode conformable epidermal sensor compared against an M-mode echocardiogram. The disclosed stacked-design multi-mode conformable epidermal sensor and echocardiogram were synchronized using the ECG signal captured on both systems. From this comparison, the AO clearly corresponds to the first major peak of the first heart sound, and the AC is near the beginning of the first major minima of the second heart sound. This result agrees with previous literature. In addition to measuring the valvular activity of the heart, the accelerometer can be used to determine the rough orientation of the user by analyzing the low-frequency (<0.5 Hz) components. The general orientations while lying supine, standing upright, and lying on the side are detected as shown in FIG. 10C. Segments of substantial motion, occurring mostly during posture change, can also be detected and are marked with a lighter-color vertical band in FIG. 10C. To test the performance of the SCG sensor, a synthetic voltage and vibration-generating phantom for replicating human-like ECG and SCG signals was used. Using this system, the quality of collected SCG signals against the known signal input to the phantom is evaluated. Both synthetic SCG signals (FIG. 10D) and real human SCG signals (FIG. 10E) were fed into the generator and were accurately reproduced by the disclosed stacked-design multi-mode conformable epidermal sensor.

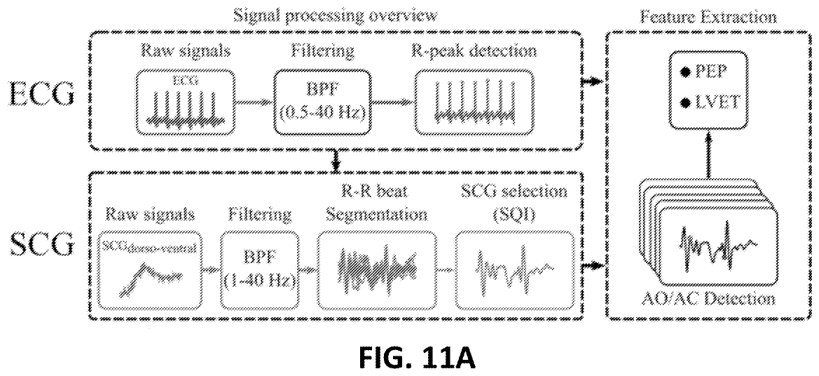

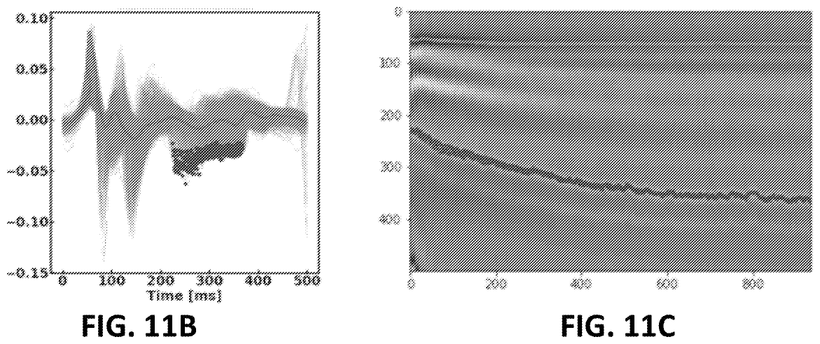

A block diagram of the pre-processing pipeline designed for extracting SCG features is shown in FIG. 11A. First, the R-peaks from the ECG signals are extracted after filtering the ECG using a band-pass filter with 0.5-40 Hz cutoff frequencies. The SCG signals are then filtered with a band-pass filter [1-40 Hz] and the SCG signals are segmented beat-by-beat using the extracted R-peaks. Next, a signal quality indexing (SQI) method for SCG beats is applied to exclude the beats corrupted by noise above a certain threshold. In one example, the SQI the exponential factor (λ) was set to 25 and the threshold to 0.5. Finally, the AO and AC time locations (FIGS. 11B, 11C) are extracted using an SCG peak tracking algorithm (see, for example, A. H. Gazi, S. Sundararaj, A. B. Harrison, N. Z. Gurel, M. T. Wittbrodt, M. Alkhalaf, M. Soudan, O. Levantsevych, A. Haffar, A. J. Shah, V. Vaccarino, J. D. Bremmer, O. T. Inan, In 43rd Annual International Conference of the IEEE Engineering in Medicine & Biology Society (EMBC). 2021 1444-1447, which is fully incorporated by reference). The heart rate (HR), PEP, and LVET are calculated using the extracted R-peak, AO, and AC timings.

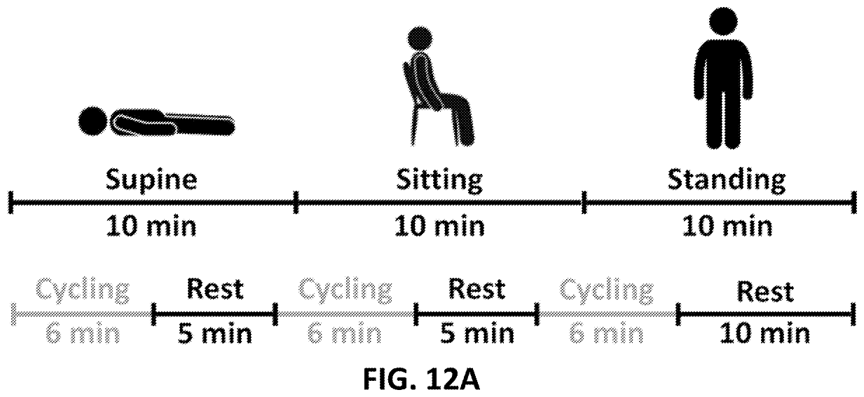

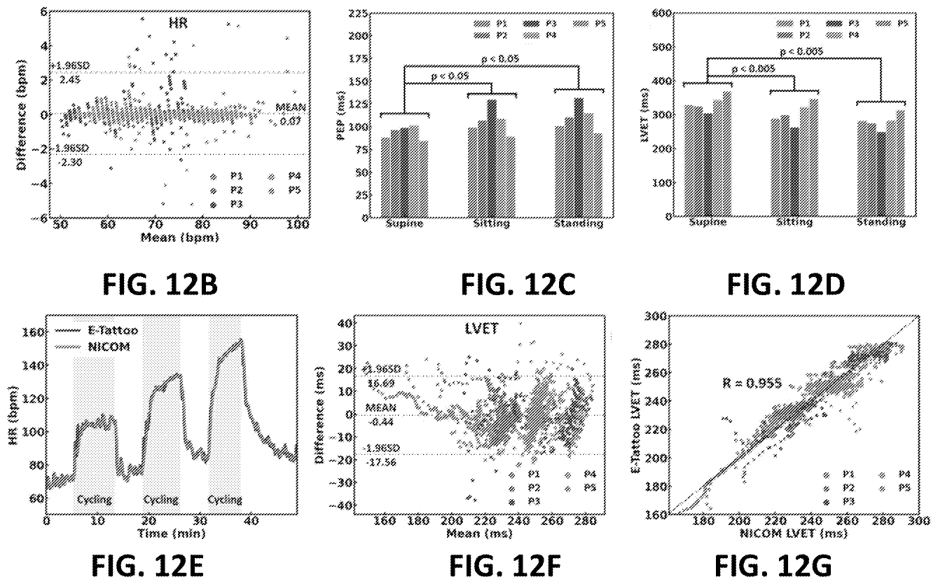

To test the disclosed stacked-design multi-mode conformable epidermal sensor, a set of experiments on 6 human subjects were conducted, split into two groups of 5 and 1 for two sets of experiments. All experiments were done under an Institutional Review Board (IRB) approved protocol at the University of Texas at Austin with IRB ID STUDY00002313. Informed consent was obtained from all subjects involved in the study. The first set of experiments, conducted on 5 participants, consisted of holding static poses and performing cycling under incremental load with breaks (FIG. 12A). Subjects simultaneously wore the disclosed stacked-design multi-mode conformable epidermal sensor along with a non-invasive cardiac output monitor (NICOM) (Starling Fluid Management Monitoring System, Baxter). The NICOM was used to measure multiple hemodynamic parameters including HR and LVET which was compared against data from the disclosed stacked-design multi-mode conformable epidermal sensor. The Bland Altman plot in FIG. 12B comparing HR from the disclosed stacked-design multi-mode conformable epidermal sensor and the NICOM during the static poses experiment shows a strong agreement for all participants with a difference of 0.07±1.21 beats per minute (bpm). FIGS. 12C and 12D show the change in average PEP and LVET respectively, measured in milliseconds (ms), for different participants as they transitioned from lying supine to sitting upright and then to standing. The increase in PEP and the decrease in LVET is well documented in prior literature, and is attributed to changes in preload, contractility, and afterload. Statistically significant differences in both PEP (p<0.05, paired t-test) and LVET (p<0.005, paired t-test) were observed when the subjects transitioned from supine to both upright and standing. This demonstrates the capability of the disclosed stacked-design multi-mode conformable epidermal sensor to measure small changes in STIs caused by posture changes. FIG. 12E shows the HR from the disclosed stacked-design multi-mode conformable epidermal sensor and from the NICOM during the cycling exercise experiment. HR from the disclosed stacked-design multi-mode conformable epidermal sensor is highly correlated to HR from the NICOM with a difference of 0.02±1.24 bpm. Thanks to the high conformability of the disclosed stacked-design multi-mode conformable epidermal sensor, the ECG signal remains pristine during cycling.

STIs were computed during the rest segments after every cycling segment. The rest segments were significantly less corrupted by motion artifacts while containing important hemodynamic recovery information. FIG. 12F shows the Bland-Altman plot comparing the LVET from the disclosed stacked-design multi-mode conformable epidermal sensor and NICOM for each participant for the entire cycling experiment. Each participant exhibited different cardiovascular responses to exercise as is evident from the variation from the resting LVET. The mean difference of LVET between the two devices is −0.44±8.74 ms. The disclosed stacked-design multi-mode conformable epidermal sensor's accuracy exceeds that of other relevant works in this field as seen in Table 1.

TABLE 1
Accuracy comparison of measuring left ventricular ejection
time (LVET) reported in the literature to the e-tattoo.

| | | No. of | | LVET error | |
|---|---|---|---|---|---|
| Reference | Ground Truth | Subjects | Wearable | Mean (ms) | SD (ms) |
| This work | NICOM (ICG) | 5 | Yes | 0 | 9 |
| [50] | Aortic Doppler | 13 | No | −14 | 9 |
| [51] | Tissue Doppler | 85 | No | −4 | 14 |
| [52] | ICON-Core (ICG) | 10 | Yes | 22 | 24 |
| [53] | Echo Doppler | 46 | No | 0 | 40 |
| [54] | Phonocardiogram | 10 | No | 5 | 86 |

SD: standard deviation, ms: milliseconds, ICG: impedance cardiogram

Where the other works referenced in Table 1 are, and are each fully incorporated by reference:
[50] G. S. Chan, P. M. Middleton, B. G. Celler, L. Wang, N. H. Lovell, Physiological measurement 2007, 28, 4 439.
[51] P. Dehkordi, F. Khosrow-Khavar, M. Di Rienzo, O. T. Inan, S. E. Schmidt, A. P. Blaber, K. Sørensen, J. J. Struijk, V. Zakeri, P. Lombardi, M. M. H. Shandhi, M. Borairi, J. M. Zanetti, K. Tavakolian, Frontiers in physiology 2019, 10 1057.
[52] M. Klum, M. Urban, T. Tigges, A.-G. Pielmus, A. Feldheiser, T. Schmitt, R. Orglmeister, Sensors 2020, 20, 7 2033.
[53] Y.-F. Yang, Y.-S. Chou, J.-Y. Wang, In International Conference on Biomedical and Health Informatics. Springer, 2019 363-370.
[54] S.-H. Liu, J.-J. Wang, C.-H. Su, D.-C. Cheng, Sensors 2018, 18, 9 3036.

FIG. 12G shows the correlation plot between the LVET measurements from the disclosed stacked-design multi-mode conformable epidermal sensor and the NICOM with a Pearson correlation coefficient of 0.955. This shows a very linear, one-to-one relationship between the measurements from the two devices. This demonstrates that the disclosed stacked-design multi-mode conformable epidermal sensor can be a viable alternative to such bulky and expensive clinical monitors for measuring STIs.

In some instances, motion artifacts may be removed from signals. Referred to herein as Filtering Averaging and Decomposition framework or FAD framework, it allows for the extraction of LVET even during sustained physical activities. For example, The ECG signal may be collected at 125 Hz and may or may not be up sampled to a higher frequency (1000 Hz., for example). The SCG signal can also be up sampled to a higher frequency (1000 Hz). The ECG and SCG signals are band-pass filtered in the frequency range of interest. ECG is filtered using a band-pass filter with cutoff frequencies values that can be changed according to the characteristics of the signal (e.g., the HR) to reduce noise and enhance the R-peak. The SCG signal is filtered with a band-pass filter using a lower cutoff frequency anywhere between 5-12 Hz and a higher cutoff frequency between 25-40 Hz. The lower cutoff frequency removes some artifacts due to motion which are present at lower frequency ranges. R-peaks are identified on the ECG signal using a peak-detection algorithm including, but not limited to Pan-Tompkins detector, matched filters, stationary wavelet, autocorrelation based, and Hamilton detectors. The SCG signal is segmented into individual frames beat-by-beat using the extracted ECG R-peaks. A window of a specified number of beats is slid forward with a certain overlap percentage (between 10% and 50%) between adjacent windows. The value of the beats will be chosen according to the requirements related to the response time of the system (the lower the number of beats the faster the response of the system). The collection of extracted frames per each window is averaged to obtain ensemble-average SCG traces, which are less noisy compared to the original heartbeats.

A signal quality index (SQI) method may or may not be applied to discard SCG beats of low quality due to noise corruption. This ensemble averaging process, using a sliding window, is iterated to segment the SCG signal into averaged traces. At this point, signal decomposition techniques, for example empirical mode decomposition (EMD), are applied for the removal of motion artifacts, which may vary depending on the recognized activity being performed. Note that the use of EMD above is not intended to limit the scope of this disclosure, and other techniques are contemplated. For example, noise reduction algorithms, such as those based on wavelets, principal component analysis, moving average filtering, independent component analysis, etc., may be used in place of EMD. These techniques eliminate noise components from the SCG signal and allow only the components of interest to pass through; motion cancellation or adaptive filtering techniques that exploit other motion sensors (e.g., accelerometers) to record the subject's movements may also be used individually, or in combination with those techniques listed above, to cancel motion artifacts from the SCG signal. As one example, for the cycling activity, EMD may or may not be applied to each ensemble-averaged trace to improve the reduction of motion artifact corruption in the SCG signals during motion. EMD is eventually applied to each ensemble-average heartbeat. EMD is applied to obtain the intrinsic mode functions (IMFs) that constitute the signal. The first or second IMFs are expected to resemble the signal and thus will be used in the further processing steps. Dynamic time warping may or may not be used to assess the improvement in signal quality between the first and/or second IMFs and the signal before EMD denoising. The IMF that has the lowest warping distance will be employed for further processing.

The FAD framework leverages the implementation of adaptive filters, ensemble averaging, and signal decomposition (e.g., Empirical Mode Decomposition (EMD)) techniques in a sequential stack for motion compensation. Adaptive filters are a category of filters that automatically modify their parameters based on input signal characteristics, proving beneficial in scenarios with evolving signal properties. Continuously adjusting coefficients to minimize the difference between actual and desired outputs enables adaptive filters to effectively monitor and respond to changes in the input signal. Various adaptive filters may be used, including Recursive Least Squares (RLS), Least Mean Squares (LMS) filters, and Normalized Least Mean Squares (NLMS) filters.

The LMS Algorithm emerges as a straightforward and easily applicable solution, with simplicity as its key advantage. Its ease of implementation and low computational complexity make it particularly well-suited for real-time applications. The LMS algorithm relies on a gradient-based approach for weight updates and does not consider past data. However, it is essential to acknowledge its slower convergence and the tendency to exhibit a higher steady-state error concerning the unknown system. NLMS ensures numerical stability by scaling filter coefficients, particularly valuable in scenarios with varying signal magnitudes. In noise and motion artifact compensation, NLMS adeptly filters unwanted disturbances while preserving essential signal components. Its value is particularly evident in applications where input signal characteristics undergo dynamic changes, such as noise and motion compensation in biomedical signal processing.

The EMD-based denoising approach has been successfully employed to yield a statistically significant improvement in the signal-to-noise ratio of wearable SCG signals. EMD is an analytical and adaptive method developed for the analysis of non-stationary signals. It involves breaking down or decomposing a signal into components that are specific only to the signal from which they are generated. Specifically, EMD decomposes a non-stationary signal into a set of zero-mean, amplitude-modulated, and frequency-modulated tones called intrinsic mode functions (IMFs). This process of decomposition is particularly advantageous in the context of SCG signal extraction during motion, where the signals are inherently non-linear and affected by a plethora of physiological and external factors. Each IMF derived from the EMD process captures a distinct oscillatory mode inherent to the original SCG signal, allowing for granular analysis of its components. The adaptability of EMD is key; it does not rely on a predetermined basis set, unlike Fourier or wavelet transforms, making it uniquely suited for handling the unpredictable nature of motion-corrupted SCG signals. Moreover, the EMD method facilitates effective noise reduction in SCG signals. By isolating and excluding those IMFs that represent noise or artifacts (often those associated with higher frequencies or irregular patterns) the denoised SCG signal retains its essential cardiac information with enhanced clarity.

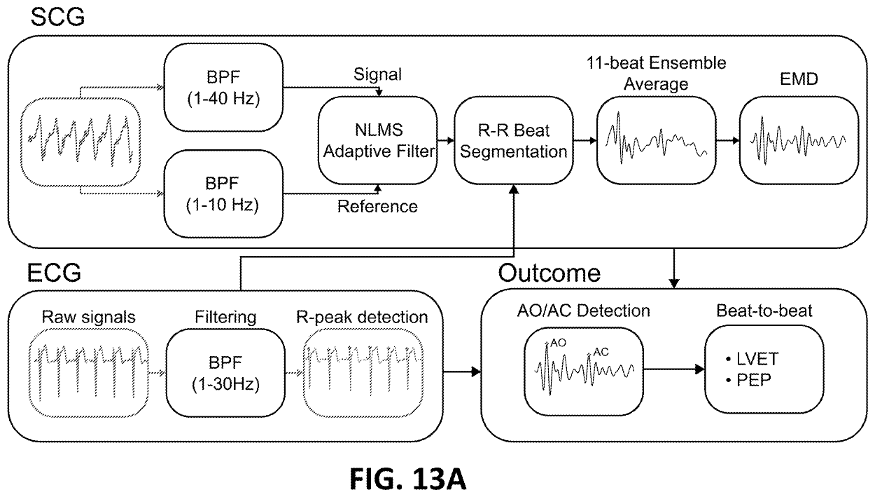

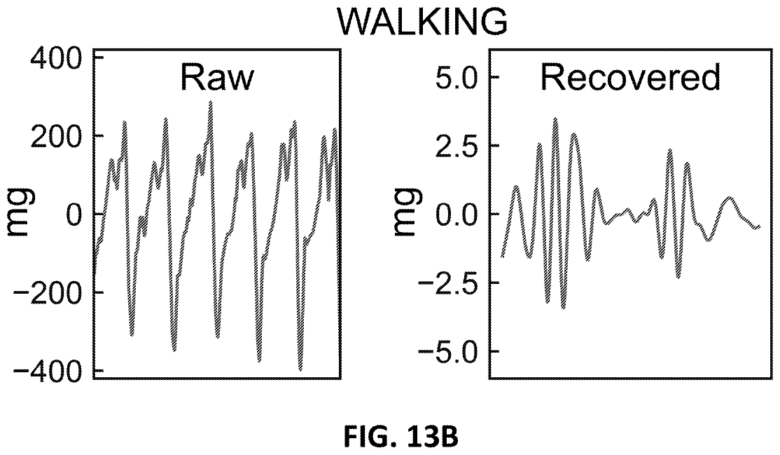

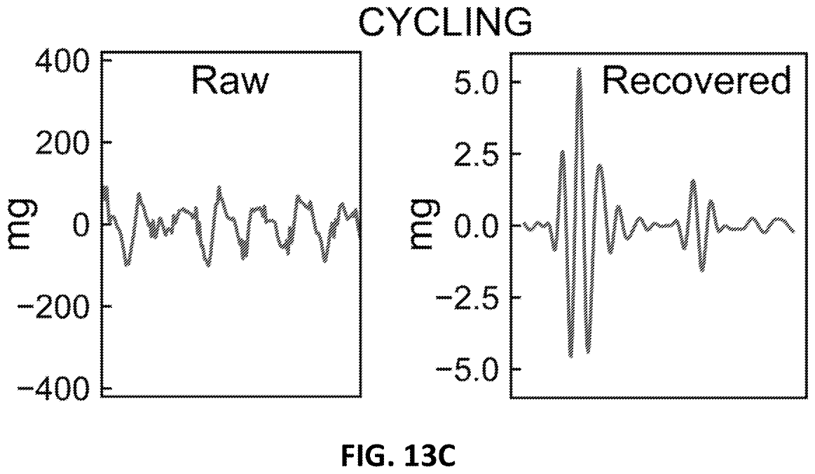

The FAD framework incorporates a multi-stage filtering approach for recovering the SCG signal during motion as illustrated in FIG. 13A. Initially, an adaptive filter is applied, followed by beat-ensembling using the ECG R-peak as the reference point. Subsequently, EMD is employed, and specific peaks are extracted from the first IMF. PEP and L VET are computed based on these extracted features, corresponding to AO and AC.

Conventional adaptive filters use two signals, one is the actual input signal to be filtered the other is the desired signal. These two signals may or may not be generated from two different sources (sensors). In some instances of the disclosed pre-processing phase, the accelerometer signal (from the accelerometer used by the SCG sensor) is used to generate two different signals using two different bandpass filters. A first, motion signal is generated using a bandpass filter (BPF) in the range of 1-10 Hz. In general, this desired motion signal can be generated using a BPF with the lower cutoff frequency in the range of 0.5-1.5 Hz and the upper cutoff frequency in the range of 5-15 Hz. In one aspect as disclosed herein, the input signal is generated with a BPF in the range 1-40 Hz. In general, the input signal can be generated with a BPF with lower cutoff frequency in the range 0.5-1.5 Hz, and the upper cutoff frequency in the range of 35-45 Hz. In the pre-processing pipeline, both signals (motion signal and input signal) are resampled to a higher sampling frequency (1000 Hz, as an example) for greater temporal resolution and then fed into the adaptive filter. The filter error is thus regarded as the output that is further processed.

In one aspect, an adaptive NLMS filter is employed to remove substantial motion artifacts from the SCG signals. The filter is implemented with a window length ‘N’ of 128 samples, a learning rate coefficient ‘u’ set at 0.7, and initially randomized weights. These and other tunable parameters of the filter can eventually be adjusted to enhance the framework's performance on different activities. The output from the NLMS filter is segmented into individual beats using the ECG signal as a reference for segmentation. Ensemble averaging is performed over a number of beats (for example, every 11 beats) with a beat overlap (for example, 8 beats) to enhance the signal-to-noise ratio (SNR), considering that the number of heartbeats required in the averaging operation is directly proportional to the walking speed. Following ensemble averaging, signal decomposition (e.g., EMD) is applied to each resulting ‘beat’ to extract the first IMF. This signal is subsequently processed to extract cardiac events of interest.

On the first IMF of each ensemble, the peaks corresponding to AO and AC are sought. Initially, the expected positions of these peaks are set. All the peaks in this signal segment are detected and the algorithm selects the nearest peaks to the expected positions. The AO and AC peak positions are then appended to arrays, and the expected positions are updated using a weighted averaging approach.

The identified AO and AC peaks are also plotted and manually verified by a human operator. To confirm the correct positioning of the AC peak, the T-wave of the ECG signal is taken as a reference. Following the peak detection process, calculations are performed to determine the PEP and LVET over time based on the detected peaks.

FIG. 13B provides a visual representation of both the raw signals and the outcomes achieved in recovering the SCG signal during two distinct activities from a single participant: walking at 3 mph and cycling (FIG. 13C) at 75 W. 3 seconds of raw data are showcased together with a 700 ms segment of the processed signal depicting one heartbeat. In the initial raw signal captured from the participant, the presence of motion artifacts dominates, rendering the mechano-acoustic cardiac signal nearly imperceptible. However, the FAD framework is effective in canceling out the motion induced component of the signal, resulting in the successful retrieval of the SCG beats. This achievement underscores the robustness and efficacy of our methodology in mitigating motion interference and facilitating the accurate extraction of SCG data even during sustained motion.

HR obtained from the NICOM device was compared to the HR derived from the R-R interval extracted from the ECG signal captured by the disclosed stacked-design multi-mode conformable epidermal sensor. The resulting data demonstrated a notably high correlation (R=0.997) and accuracy (error=0.13±3.72) across all subjects. This correlation not only attests to the precision of the disclosed stacked-design multi-mode conformable epidermal sensor's HR measurements, but also implies that the majority of R-peaks were accurately detected. This accuracy in R-peak detection holds significant importance in ensuring correct beat-to-beat segmentation and ensemble averaging required for the reliable operation of the FAD framework.

The aortic valve opening (AO) and aortic valve closing (AC) time locations are extracted using a signal peak detection algorithm based on some parameters such as causal temporal and/or amplitude thresholds. Based on AO and AC-identified peaks and on R-peaks locations, pre-ejection period (PEP) and left ventricular ejection time (LVET) intervals are computed for each window. Each PEP and LVET value is associated with a time instant. The time value for each window is chosen as the mean of the time index for the first and last heartbeat in the window considered. Savitsky-Golay filtering using a third or lower-degree polynomial may be fitted to the estimates of PEP and LVET to display the trend and perform denoising. Note that the proposed algorithm has also the potential to allow the extraction of the heart rate (i.e., HR) and Left Ventricular Ejection Fraction (i.e., LVEF), which is a measure of the heart's efficiency in pumping blood and it is useful for guiding treatment decisions in patients with heart disease. HR may be extracted either from the ECG or SCG signal, based on the identified R or AO peaks respectively, or using any time/frequency domain method for HR estimation.

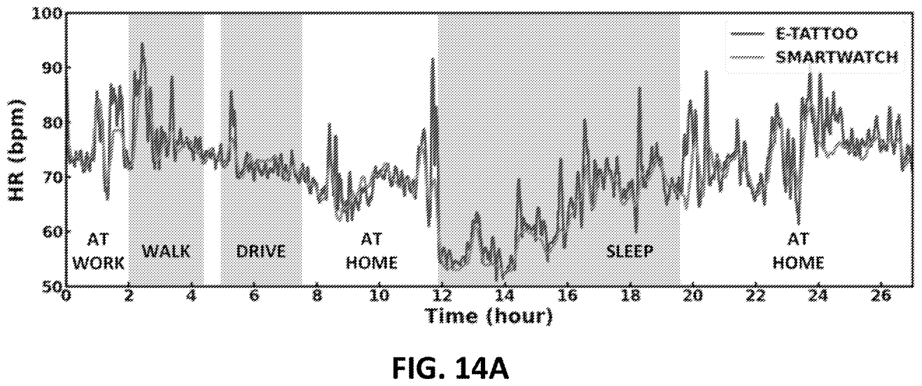

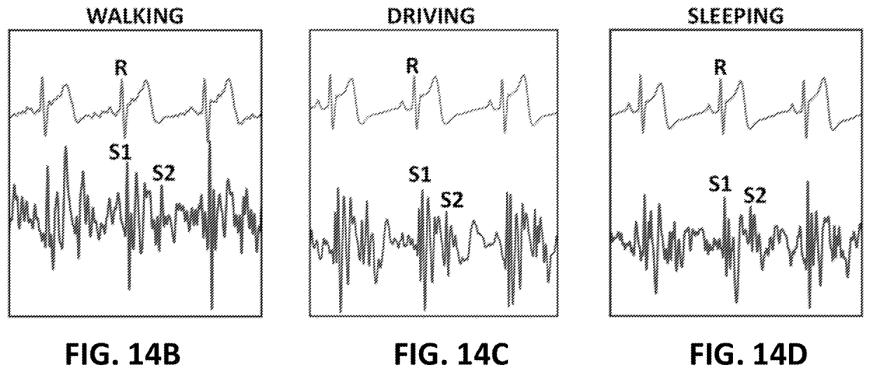

For the last experiment, a long-term ambulatory study was performed on one participant for more than 24 hrs. Due to the non-portability of the NICOM and the lack of any wearable LVET or PEP measuring devices, the disclosed stacked-design multi-mode conformable epidermal sensor's long-term LVET and PEP data could not be compared against a reference device. The goal of the study was to validate if embodiments of the disclosed stacked-design multi-mode conformable epidermal sensor can be unobtrusive, and minimally obstructive wearable devices that can be worn in day-to-day lives and in most social settings. Keeping this in mind, a Samsung Galaxy Watch 3 was used for long-term HR comparison (see FIG. 14A). The long-term data was manually inspected to find segments of ECG and SCG data collected during different activities, and it can be observed in FIGS. 12B-12D. These segments are ideal for extracting STIs and contextualizing them to the activity being performed would provide a more holistic understanding of the cardiovascular health of the user. Sleep monitoring is also a very important aspect as motion artifacts are kept to a minimum and sleep hemodynamics offer unique insight into cardiovascular regulation mechanisms in pathological conditions associated with sleep disorders such as sleep apnea and insomnia. This long-term study showcased the disclosed stacked-design multi-mode conformable epidermal sensor as viable unobtrusive device for cardiovascular monitoring.

In the specification and/or figures, typical embodiments have been disclosed. The present disclosure is not limited to such exemplary embodiments. Those skilled in the art will also appreciate that various adaptations and modifications of the preferred and alternative embodiments described above can be configured without departing from the scope and spirit of the disclosure.

The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A stacked-design multi-mode conformable epidermal sensor, comprising:
a transparent first flexible, stretchable insulating substrate comprising a first side and a second side opposite the first side, wherein the first side adheres to an epidermis and the transparent first flexible, stretchable insulating substrate conforms to the epidermis;
an electrocardiogram (ECG) sensor comprising one or more biocompatible electrodes, wherein each electrode forms an electrode pattern on the first side of the transparent first flexible, stretchable insulating substrate and each electrode is configured to flex and stretch with the transparent first flexible, stretchable insulating substrate to conform to the epidermis;
a seismocardiogram (SCG) sensor, said SCG sensor mounted on a second side of a second flexible insulating substrate, said second flexible insulating substrate having a first side that is substantially in contact with the second side of the transparent first flexible, stretchable insulating substrate; and
electronics disposed at least partially on the second side of the second flexible insulating substrate, said electronics comprising at least a wireless communications interface, a central processing unit, an accelerometer, the ECG sensor, and a power source, wherein components of the electronics are separated from one another on the second side of the second flexible insulating substrate and connected to one another using copper traces configured to flex and stretch with the transparent first flexible, stretchable insulating substrate and/or the second flexible insulating substrate, and to conform to the epidermis, wherein the electronics are connected to the one or more biocompatible electrodes of the ECG sensor using conductive material in the transparent first flexible, stretchable insulating substrate, or conductive material in both the transparent first flexible, stretchable insulating substrate and the second flexible insulating substrate.

2. The stacked-design multi-mode conformable epidermal sensor of claim 1, further comprising: a photoplethysmogram (PPG) sensor disposed on the first side of the second flexible insulating substrate, said PPG sensor comprising one or more light emitting diodes (LEDs) and one or more photodetectors (PDs) that perform reflective photoplethysmography through the transparent first flexible, stretchable insulating substrate, said one or more LEDs and the one or more PDs electrically connected to the electronics disposed at least partially on the second side of the second flexible insulating substrate through the second flexible insulating substrate; and
a temperature sensor, said temperature sensor disposed on the first side of the second flexible insulating substrate and connected to the electronics disposed at least partially on the second side of the second flexible insulating substrate through the second flexible insulating substrate.

3. (canceled)

4. (canceled)

5. (canceled)

6. The stacked-design multi-mode conformable epidermal sensor of claim 1, wherein the electronics further comprise a second accelerometer, wherein the second accelerometer is used for one or more of motion compensation, orientation detection, and activity recognition.

7. (canceled)

8. The stacked-design multi-mode conformable epidermal sensor of claim 2, wherein one or more of the ECG sensor, the SCG sensor, the PPG sensor, and the temperature sensor are electrically connected to one another and synchronized, and wherein signals from one or more of the ECG sensor, the SCG sensor, the PPG sensor, and the temperature sensor of the stacked-design multi-mode conformable epidermal sensor are synchronized with one another and/or with the wireless communications interface and/or the central processing unit of the stacked-design multi-mode conformable epidermal sensor.

9. (canceled)

10. The stacked-design multi-mode conformable epidermal sensor of claim 8, wherein the second flexible insulating substrate is patterned in an island-serpentine design and the electronics comprise islands of densely packed electronics connected by stretchable serpentine interconnects.

11. (canceled)

12. The stacked-design multi-mode conformable epidermal sensor of claim 1, wherein the one or more biocompatible electrodes of the ECG sensor and the transparent first flexible, stretchable insulating substrate can be separated from the electronics and the second flexible insulating substrate and discarded such that the electronics and the second flexible insulating substrate can be reused to form a second multi-mode epidermal sensor.

13. (canceled)

14. (canceled)

15. (canceled)

16. (canceled)

17. (canceled)

18. (canceled)

19. (canceled)

20. The stacked-design multi-mode conformable epidermal sensor according to claim 1, wherein the electrode pattern on the first side of the transparent first flexible, stretchable insulating substrate is serpentine shaped, and wherein each electrode pattern includes one or more terminal pads for connection to an interconnect.

21. (canceled)

22. (canceled)

23. (canceled)

24. The stacked-design multi-mode conformable epidermal sensor according to claim 1, further comprising motion artifact removal of signals generated by the SCG sensor, wherein the motion artifact removal comprises an implementation of an adaptive filter, ensemble averaging, and signal decomposition techniques in a sequential stack for motion compensation, wherein the implementation of the adaptive filter comprises one or more of Recursive Least Squares (RLS) filters, Least Mean Squares (LMS) filters, and Normalized Least Mean Squares (NLMS) filters, and
wherein a signal from the accelerometer generates two different signals using bandpass filters; said two different signals comprising a motion signal and an input signal, wherein both the motion signal and the input signal are resampled to a higher sampling frequency for greater temporal resolution and then fed into the adaptive filter, said adaptive filtering having an output comprising filter error, wherein the motion signal is generated using a first bandpass filter in a range of approximately 1-10 Hz, and wherein the first bandpass filter has a lower cutoff frequency in a range of approximately 0.5-1.5 Hz and an upper cutoff frequency in a range of approximately 5-15 Hz.

25. (canceled)

26. (canceled)

27. (canceled)

28. (canceled)

29. The stacked-design multi-mode conformable epidermal sensor according to claim 24, wherein the input signal is generated using a second bandpass filter in a range of approximately 1-40 Hz, wherein the second bandpass filter has a lower cutoff frequency in a range 0.5-1.5 Hz, and an upper cutoff frequency in a range of approximately 35-45 Hz.

30. (canceled)

31. The stacked-design multi-mode conformable epidermal sensor according to claim 24, wherein the motion and input signals are resampled to the higher sampling frequency of approximately 1000 Hz.

32. (canceled)

33. A method for using a stacked-design multi-mode conformable epidermal sensor, the method comprising:
attaching a stacked-design multi-mode conformable epidermal sensor to an epidermis, said stacked-design multi-mode conformable epidermal sensor comprising:
a transparent first flexible, stretchable insulating substrate comprising a first side and a second side opposite the first side, wherein the first side adheres to the epidermis and the transparent first flexible, stretchable insulating substrate conforms to the epidermis;
an electrocardiogram (ECG) sensor comprising one or more biocompatible electrodes, wherein each electrode forms an electrode pattern on the first side of the transparent first flexible, stretchable insulating substrate and each electrode is configured to flex and stretch with the transparent first flexible, stretchable insulating substrate to conform to the epidermis;
a seismocardiogram (SCG) sensor, said SCG sensor mounted on a second side of a second flexible insulating substrate, said second flexible insulating substrate having a first side that is substantially in contact with the second side of the transparent first flexible, stretchable insulating substrate; and
electronics disposed at least partially on the second side of the second flexible insulating substrate, said electronics comprising at least a wireless communications interface, a central processing unit, an accelerometer, and a power source, wherein components of the electronics are separated from one another on the second side of the second flexible insulating substrate and connected to one another using conductive traces configured to flex and stretch with the transparent first flexible, stretchable insulating substrate and/or the second flexible insulating substrate, and to conform to the epidermis, wherein the electronics are connected to the one or more biocompatible electrodes of the ECG sensor using conductive material in the transparent first flexible, stretchable insulating substrate, or conductive material in both the transparent first flexible, stretchable insulating substrate and the second flexible insulating substrate; and
connecting ECG test equipment to the ECG sensor and SCG test equipment to the SCG sensor; and
simultaneously measuring at least an electrocardiogram and seismocardiogram using the ECG test equipment and the SCG test equipment.

34. The method of claim 33, wherein the stacked-design multi-mode conformable epidermal sensor further comprises a photoplethysmogram (PPG) sensor disposed on the first side of the second flexible insulating substrate, said PPG sensor comprising one or more light emitting diodes (LEDs) and one or more photodetectors (PDs) that perform reflective photoplethysmography through the transparent first flexible, stretchable insulating substrate, said one or more LEDs and the one or more PDs electrically connected to the electronics disposed at least partially on the second side of the second flexible insulating substrate through the second flexible insulating substrate; and
a temperature sensor, said temperature sensor disposed on the first side of the second flexible insulating substrate and connected to the electronics disposed at least partially on the second side of the second flexible insulating substrate through the second flexible insulating substrate.

35. (canceled)

36. The method of claim 34, wherein signals from one or more of the ECG sensor, the SCG sensor, the PPG sensor, and the temperature sensor of the stacked-design multi-mode conformable epidermal sensor are synchronized with one another and/or with the wireless communications interface and/or the central processing unit of the stacked-design multi-mode conformable epidermal sensor, and wherein signals from one or more of the ECG sensor, the SCG sensor, the PPG sensor, and the temperature sensor of the stacked-design multi-mode conformable epidermal sensor are synchronized with one another and/or with the wireless communications interface and/or the central processing unit of the stacked-design multi-mode conformable epidermal sensor.

37. The method of claim 36, further comprising:
computing a beat-to-beat blood pressure (BP) from the electrocardiogram, seismocardiogram and photoplethysmogram;
measuring one or more cardiac timing intervals (CTIs) using the stacked-design multi-mode conformable epidermal sensor,
wherein the CTIs include a pre-ejection period (PEP) and a left ventricular ejection time (LVET), and
wherein the PEP and LVET are used to assess left ventricular (LV) performance.

38. (canceled)

39. (canceled)

40. (canceled)

41. The method of claim 33, wherein the second flexible insulating substrate is patterned in an island-serpentine design and the electronics comprise islands of densely packed electronics connected by stretchable serpentine interconnects.

42. (canceled)

43. (canceled)

44. (canceled)

45. The method according to claim 33, further comprising motion artifact removal of signals generated by the SCG sensor, wherein the motion artifact removal comprises an implementation of an adaptive filter, ensemble averaging, and signal decomposition techniques in a sequential stack for motion compensation,
wherein the implementation of the adaptive filter comprises one or more of Recursive Least Squares (RLS) filters, Least Mean Squares (LMS) filters, and Normalized Least Mean Squares (NLMS) filters,
wherein a signal from the accelerometer generates two different signals using bandpass filters; said two different signals comprising a motion signal and an input signal, wherein both the motion signal and the input signal are resampled to a higher sampling frequency for greater temporal resolution and then fed into the adaptive filter, said adaptive filtering having an output comprising filter error,
wherein the motion signal is generated using a first bandpass filter in a range of approximately 1-10 Hz,
wherein the motion signal is generated using a first bandpass filter in a range of approximately 1-10 Hz,
wherein the first bandpass filter has a lower cutoff frequency in a range of approximately 0.5-1.5 Hz and an upper cutoff frequency in a range of approximately 5-15 Hz,
wherein the input signal is generated using a second bandpass filter in a range of approximately 1-40 Hz,
wherein the second bandpass filter has a lower cutoff frequency in a range 0.5-1.5 Hz, and an upper cutoff frequency in a range of approximately 35-45 Hz, and
wherein the second bandpass filter has a lower cutoff frequency in a range 0.5-1.5 Hz, and an upper cutoff frequency in a range of approximately 35-45 Hz.

46. (canceled)

47. (canceled)

48. (canceled)

49. (canceled)

50. (canceled)

51. (canceled)

52. (canceled)

53. (canceled)
